(19) United States
(12) Patent Application Publication
(10) Pub. No.: US 2011/0184379 A1
(43) Pub. Date: Jul. 28, 2011

(54) METHOD AND SYSTEM TO DEFINE PATIENT SPECIFIC THERAPEUTIC REGIMENS BY MEANS OF PHARMACOKINETIC AND PHARMACODYNAMIC TOOLS

(75) Inventors: William P. Van Antwerp, Valencia, CA (US); Robert C. Hamlen, Edina, MN (US); Eric A. Grovender, Minneapolis, MN (US)

(73) Assignee: MEDTRONIC, INC., Minneapolis, MN (US)

(21) Appl. No.: 12/934,054

(22) PCT Filed: Mar. 27, 2009
(86) PCT No.: PCT/US09/38617

§ 371 Date: Mar. 24, 2011

Related U.S. Application Data
(60) Provisional application No. 61/040,026, filed on Mar. 27, 2008.
(60) Provisional application No. 61/040,038, filed on Mar. 27, 2008.
(60) Provisional application No. 61/058,006, filed on Jun. 2, 2008.
(60) Provisional application No. 61/058,001, filed on Jun. 2, 2008.
(60) Provisional application No. 61/197,772, filed on Oct. 30, 2008.

(51) Int. Cl. A61M 5/168; A61K 38/21; A61K 31/7056; A61P 31/14

(52) U.S. Cl. 604/503; 424/85.7; 514/43; 604/66

(57) ABSTRACT

Methods for treating Hepatitis infections are provided. In one embodiment, an initial dosage of interferon is administered to a patient, and interferon serum levels and viral load data is collected over time. This data can be used to determine patient-specific pharmacokinetic and pharmacodynamic parameters and then construct patient-specific interferon delivery profiles. Patient-specific delivery profiles can then be used to design patient-specific therapeutic regimens.

### CROSS REFERENCE TO RELATED APPLICATIONS

This application claims priority under Section 119(e) from U.S. Provisional Application Ser. No. 61/040,026 filed Mar. 27, 2008; U.S. Provisional Application Ser. No. 61/040,038 filed Mar. 27, 2008; U.S. Provisional Application Ser. No. 61/058,001 filed Jun. 2, 2008; U.S. Provisional Application Ser. No. 61/058,006 filed Jun. 2, 2008; and U.S. Provisional Patent Application Ser. No. 61/197,772 filed Oct. 30, 2008, the contents of each of which are incorporated herein by reference.

### FIELD OF THE INVENTION

This invention relates to the design of therapies for the treatment of pathological conditions (e.g. Hepatitis C virus infections). In particular, this invention relates to methods and systems for obtaining patient-specific regimen responsiveness profiles and using these profiles to optimize the therapeutic regimen(s) administered to the patient (e.g. for treatment of Hepatitis C virus infections).

### BACKGROUND OF THE INVENTION

Hepatitis C virus (HCV) infection is the most common chronic blood borne infection in the United States. Chronic liver disease is the tenth leading cause of death among adults in the United States, accounting for approximately 25,000 deaths annually, or approximately 1% of all deaths. The high prevalence of chronic HCV infection has important public health implications for the future burden of chronic liver disease in the United States. Data derived from the National Health and Nutrition Examination Survey (NHANES III) indicates that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million.

Currently, treatments for chronic hepatitis C infection typically include the administration of combinations of ribavirin and interferon-α. Ribavirin is a nucleoside analog that when incorporated into cells, interferes with viral replication (similar to action of AZT in HIV infection). It is interesting to note that while ribavirin is not effective as a stand-alone therapy for HCV, it potentiates interferon effectiveness through an as yet unknown mechanism. For example, in controlled clinical studies, ribavirin monotherapy has negligible efficacy and PEG-interferon alone has an effectiveness of 11% in a genotype 1 population. However, when ribavirin is combined with interferon-α, the therapeutic effectiveness of the combination is 29% in this population (see, e.g. Sjogren et al., Dig Dis Sci. 2005 April; 50(4):727-32, the contents of which are incorporated by reference). A variety of such therapeutic methods for the treatment of hepatitis C infection are described for example in PCT patent applications such as WO 2005/067454; WO2005/018330; WO2005/062949; WO2006/130553; WO20060130626; and WO2006/130627; United States patent applications such as 2005/0191275; US 2005/0201980; 2007/004635; US2006/281689; and 2006/276405 and articles such as Perdita, et. al., World Journal of Gastroenerology, 7(2):222-227, (April 2001); Bizollon, et. al., Hepatology, 26(2):500-504, (August 1997); Alberti, et. al. Liver Transplantation, 7(10):870-876, (October 2001); Shakil, et. al., Hepatology, 36(5):1253-1258, (November 2002); Schalm, et. al., Gut, 46:562-568, (April 2000); and Yurdaydin et al., Journal of Viral Hepatitis, 12(7):262-268, (May 2005), the contents of which are incorporated herein by reference. Unfortunately however, while great strides have been made in the treatment of HCV infection, clinical success rates are only about 50% and have progressed slowly since the introduction of interferon into the clinic (see, e.g. Smith, R., Nat Rev Drug Discov. 2006, 5(9):715-6, the contents of which are incorporated by reference).

As current clinical practices eliminate HCV in only about 50% of infected individuals, new therapies are highly desirable. The development of such therapies is complicated however by the observation that host factors such as ethnicity, obesity, insulin resistance and hepatic fibrosis, as well as viral factors such as genotype and baseline viral load, can have a profound impact on the success of a given therapeutic regimen. In addition, current therapeutic regimens last for an extended period of time and patients often suffer from a host of adverse dose-dependent side-effects including severe flu-like symptoms, which can negatively impact patient compliance and outcome. In this context, typical therapeutic regimens used in the treatment of hepatitis C infection follow standardized protocols and the pharmacokinetics and/or pharmacodynamics of such regimens are not tuned to the individualized physiology and infection profiles specific to each infected patient. Accordingly, there is a need for improved methods for treating viral infections such as hepatitis C, in particular the development of methods and systems for obtaining patient-specific regimen responsiveness profiles (e.g. those relating to a patient's individualized viral infection and interferon-α responsiveness characteristics) and the associated design of patient-specific drug delivery regimens based upon these profiles.

### SUMMARY OF THE INVENTION

The invention disclosed herein has a number of embodiments. One illustrative embodiment of the invention is a method of using a patient-specific regimen responsiveness profile obtained from a patient infected with hepatitis C virus (HCV) to design a patient-specific therapeutic regimen. This method comprises administering at least one therapeutic agent to the patient following a first therapeutic regimen and then obtaining pharmacokinetic or pharmacodynamic parameters from the patient in order to observe a patient-specific response to the first therapeutic regimen. Typical pharmacokinetic and/or pharmacodynamic parameters observed for example comprise the in vivo concentrations of the therapeutic agent in the patient that results from the first therapeutic regimen and/or the levels of hepatitis C virus RNA present in vivo (e.g. as found in blood, plasma, serum etc.). In such embodiments of the invention, practitioners can then use the pharmacokinetic or pharmacodynamic parameters observed in the patient following the first therapeutic regimen to obtain a patient-specific regimen responsiveness profile. This patient-specific regimen responsiveness profile is based upon the HCV infected patient's individualized physiology and consequently incorporates host factors such as obesity and hepatic fibrosis as well as viral factors such as the specific HCV genotype infecting the patient. This patient-specific regimen responsiveness profile can then be used to design a patient-specific therapeutic regimen, one that takes into account the host and viral factors unique to each infected individual.

Embodiments of the invention can use information obtained from patient-specific regimen responsiveness profiles to design a variety of patient-specific therapeutic regimens. In typical embodiments of the invention, the therapeutic regimen comprises interferon-α and the patient-specific therapeutic regimen is selected to modulate serum interferon-α concentrations in the patient. Optionally for example, the patient-specific therapeutic regimen is selected to maintain serum interferon-α concentrations in the patient at a value greater than a EC₅₀, a concentration at which the effectiveness of interferon-α is 50% of its maximum. Alternatively, the patient-specific therapeutic regimen is selected to maintain serum interferon-α concentrations in the patient at a value where the actual efficacy of interferon-α in the patient is greater than the critical efficacy of interferon-α. In other embodiments, the patient-specific therapeutic regimen is selected to modulate interferon-α concentrations in the patient so that the patient is administered different interferon dosing regimens during different phases of hepatitis C viral load decline. In other embodiments, the patient-specific therapeutic regimen is selected to modulate interferon-α concentrations in the patient so that a difference between the actual efficacy of interferon-α and the critical efficacy of interferon-α in the patient is increased. In other embodiments, the patient-specific therapeutic regimen is selected to modulate interferon-α concentrations in the patient so as to reduce dose-dependent side effects observed during the administration of interferon-α. In certain specific embodiments of the invention, the patient-specific therapeutic regimen is designed to maintain plasma interferon-α levels in the patient above a set-point, e.g. above 20, 30, 40, 50, 60, 70, 80, 90, 100, 110 or 120 IU/mL. Alternatively, the patient-specific therapeutic regimen can be designed to maintain plasma interferon-α levels in the patient below a set-point, e.g. below 140, 130, 120, 110, 100, 90, 80, 70 or 60 IU/mL. In certain embodiments of the invention, interferon-α is administered (e.g. using a continuous infusion pump) so as to deliver this cytokine at a rate selected to hit a pre-defined set point for epsilon, such as at least 0.5, 0.6, 0.65, 0.7, 0.75, 0.8, 0.85, 0.9, 0.95, 0.99, 0.999 etc.

Certain embodiments of the invention use algorithms to obtain pharmacokinetic or pharmacodynamic parameters that comprise the patient-specific profile. For example, in one embodiment of the invention, the first therapeutic regimen comprises interferon-α and observed parameters comprise a concentration of administered interferon-α in the serum of the patient that is obtained from the patient using an algorithm comprising:

$$\frac{d D}{d t} = Q - {k}_{a} D$$

$$\frac{d C}{d t} = ( \frac{{k}_{a}}{{V}_{d}^{′}} ) D - {k}_{e} C$$

$$or$$

$$ɛ ( t ) = \frac{{C ( t )}^{n}}{{EC}_{50}^{n} + {C ( t )}^{n}}$$

wherein:

D represent dose of interferon in the infusion site (IU);

Q represents infusion rate of interferon (IU/hour);

kₐ represent interferon absorption rate constant (1/hour);

kₑ represents interferon elimination rate constant (1/hour);

Vd′ represents apparent volume of distribution (mL);

C represents plasma concentration of interferon (IU/mL);

EC₅₀ represents concentration at which drug's efficacy is half its maximum (IU/mL);

n represents Hill's coefficient; and

ε represents actual efficacy.

In some embodiments of the invention, observed parameters comprise a concentration of hepatitis C virus in one or more in vivo compartments (e.g. the viral load) and are obtained from the patient using an algorithm comprising:

$$\frac{d T}{d t} = s + rT ( 1 - \frac{T + I}{{T}_{max}} ) - d T - β VT$$

$$\frac{d I}{d t} = β VT + rI ( 1 - \frac{T + I}{T max} ) - δ I \\ \frac{d V}{d t} = ( 1 - ɛ ) pI - cV$$

wherein:

T represents the concentration of uninfected target cells (cells/ml);

I represents the concentration of infected target cells (cells/ml);

Tₘₐₓ represents a maximum size of the liver (cells/ml)

V represents viral load (IU/ml);

s represents a constant rate of uninfected target cells production (cell ml⁻¹*day⁻¹);

r represents maximum specific proliferation rate of infected and uninfected target cells (day⁻¹);

β represents the infection rate constant (ml*day⁻¹*IU⁻¹);

p represents virion production rate constant (IU*cell⁻¹*day⁻¹);

c represents virion clearance rate constant (day⁻¹);

δ represents the specific death for infected target cells (day⁻¹);

d represents the specific death rate for uninfected target cells (day⁻¹); and

ε represents overall drug efficacy.

In some embodiments of the invention, observed parameters comprise the actual efficacy of the first therapeutic regimen and are determined using an algorithm comprising:

V(t)=V_{bar}[1−ε+εe^{-ct}]

### Wherein:

V(t) represents viral load (IU/ml);

V_{bar} represents initial viral load (IU/ml);

ε represents actual efficacy;

t represents time (day); and

c represents clearance constant (day⁻¹).

In some embodiments of the invention, observed parameters comprise the critical efficacy of the therapeutic agent in the first therapeutic regimen is determined in the patient is calculated using an algorithm comprising:

$${ɛ}_{c} = 1 - \frac{c ( δ {T}_{max} + r {\overline{T}}_{0} - {rT}_{max} )}{p β {T}_{max} {\overline{T}}_{0}}$$

wherein T̅ₒ is a number of uninfected target cells at uninfected steady state (I=V=0) which may be represented as:

$${\overline{T}}_{o} = \frac{{T}_{max}}{2 r} [ r - d + \sqrt{{( r - d )}^{2} + \frac{4 rs}{{T}_{max}}} ]$$

wherein r>d and s≦dTₘₐₓ so T̅ₒ≦T_{max.}

The patient-specific profiles of the invention can be used to design patient specific therapeutic regimens for use in a number of contexts. For example, in embodiments of the invention where the profile includes assessments of HCV infected cells, the patient-specific therapeutic regimen can be initiated or modulated when the ratio of the number of HCV uninfected target cells to the number of HCV infected cells is greater than or equal to 1. In embodiments of the invention where the profile includes an assessment of plasma interferon-α concentrations, the patient-specific therapeutic regimen can comprise administering a dose of interferon-α for a period of time selected to maintain a plasma interferon-α concentration above (or below) a set-point for that period of time. In certain embodiments of the invention, the patient-specific profile can be used to generally assess the patient's likely virological response to a defined interferon-α composition (e.g. pegylated or non-pegylated interferon-α) or a defined interferon-α dosing regimen.

In typical embodiments of the invention, the patient-specific therapeutic regimen comprises administering interferon-α using a continuous infusion pump. Optionally the therapeutic regimen further comprises an additional anti-viral agent such as ribavirin, VX-950; SCH 503034; R1626; or R71278. In certain embodiments of the invention, the patient-specific therapeutic regimen comprises administering a first dose of interferon-α (and/or ribavirin) during a first phase of hepatitis C viral decline and a second dose of interferon-α (and/or ribavirin) during a second phase of hepatitis C viral decline.

Once a first patient-specific regimen is designed and administered, practitioners can then obtain a further patient-specific regimen responsiveness profile that results from the administration of the first patient-specific regimen. Such further patient-specific regimen responsiveness profile can then be used to design further patient-specific regimens so that the patient's therapy continues to be precisely tailored to the host and viral factors that are unique to the patient and which fluctuate over the course of the patient's comprehensive therapy. For example, certain embodiments of the invention comprise obtaining pharmacokinetic or pharmacodynamic parameters from the patient so as to observe a patient-specific response to the first patient-specific therapeutic regimen as discussed above, wherein the pharmacokinetic or pharmacodynamic parameters comprise at least one of: a concentration of administered interferon-α in the plasma of the patient; or a concentration of hepatitis C virus in the plasma of the patient; using the pharmacokinetic or pharmacodynamic parameters observed in the patient in response to the first patient-specific therapeutic regimen to obtain a second patient-specific regimen responsiveness profile; and using the second patient-specific regimen responsiveness profile to design a second (or third or fourth etc.) patient-specific therapeutic regimen.

Certain embodiments of the invention can be implemented on a computer system. One such embodiment of the invention is a method of administering interferon-α to a patient suffering from a Hepatitis C infection, the method comprising: administering interferon-α to the patient following a first therapeutic regimen; obtaining pharmacokinetic or pharmacodynamic parameters from the patient to observe a patient-specific response to the first therapeutic regimen programmed into a controller that operably coupled to a continuous infusion pump. The continuous infusion pump having this program can then be used to administer interferon-α to the patient according to the controller programming. In one such computer implemented embodiment of the invention, the controller is programmed so that the continuous infusion pump administers interferon-α in a manner that: maintains serum interferon-α concentrations in the patient at a value greater than a EC₅₀ and/or a concentration at which the effectiveness of interferon-α is 50% of its maximum; and/or maintains serum interferon-α concentrations in the patient at a value where the actual efficacy of interferon-α in the patient is greater than the critical efficacy of interferon-α; and/or modulates interferon-α concentrations in the patient so that the patient is administered different interferon dosing regimens during different phases of hepatitis C viral load decline; and/or modulates interferon-α concentrations in the patient so that a difference between the actual efficacy of interferon-α and the critical efficacy of interferon-α in the patient is increased; and/or modulates interferon-α concentrations in the patient so as to reduce adverse side effects observed during the administration of interferon-α. In other embodiments of the invention, the continuous infusion pump administers interferon-α so as to deliver this cytokine at a rate selected to hit a pre-defined set point for epsilon, such as at least 0.5, 0.6, 0.65, 0.7, 0.75, 0.8, 0.85, 0.9, 0.95, 0.99, 0.999 etc. In another computer implemented embodiment of the invention, the controller is programmed so that the continuous infusion pump administers interferon-α at a dose and for a period of time selected to maintain a plasma interferon-α concentration above (or below) a set-point for the period of time; and the patient-specific therapeutic regimen further comprises administering a nucleoside analog that interferes with Hepatitis C viral replication (e.g. ribavirin).

A related embodiment of the invention is a system for administering interferonα to a patient having a hepatitis C infection, the system comprising: a continuous infusion pump having a medication reservoir comprising interferon-α; and a processor operably connected to the continuous infusion pump that comprises a set of instructions that causes the continuous infusion pump to administer the interferon-α to the patient according to a patient-specific therapeutic regimen made according to an embodiment of the invention. In certain embodiments of this system the interferon-α delivered by this continuous infusion pump is not conjugated to a polyol. In some embodiments of this system, the continuous infusion pump has dimensions smaller than 15×15 centimeters. Optionally the continuous infusion pump is operably coupled to an interface that facilitates the patient's movements while using the continuous infusion pump, wherein the interface comprises a clip, a strap, a clamp or a tape.

Yet another embodiment of the invention is a program code storage device, comprising: a computer-readable medium; a computer-readable program code, stored on the computer-readable medium, the computer-readable program code having instructions, which when executed cause a controller operably coupled to a medication infusion pump to administer the interferon-α to a patient infected with the hepatitis C virus according to a patient-specific therapeutic regimen made by: administering interferon-α to the patient following a first therapeutic regimen obtaining pharmacokinetic or pharmacodynamic parameters from the patient so as to observe a patient-specific response to the first therapeutic regimen wherein the pharmacokinetic or pharmacodynamic parameters comprise at least one of: a concentration of interferon-α in the blood of the patient that results from the first therapeutic regimen; or a concentration of hepatitis C virus present in the patient; using the pharmacokinetic or pharmacodynamic parameters observed in the patient in response to the first therapeutic regimen to obtain a patient-specific regimen responsiveness profile; and then using the patient-specific regimen responsiveness profile to make the patient-specific therapeutic regimen.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1A presents an exemplary generalized computer system 202 that can be used to implement elements the present invention. FIG. 1B presents one embodiment of a specific illustrative computer system embodiment that can be used with embodiments of the invention in the treatment of Hepatitis C virus infection.

FIG. 2 depicts one possible relationship between interferon concentration and interferon efficacy.

FIG. 3 depicts changes in interferon concentration over time.

FIG. 4 shows a rapid increase in efficacy from one constant rate to a higher constant rate in embodiments where the duration of stages is defined in terms of ratio of infected and uninfected target cells.

FIG. 5 presents model sensitivity to the specific death rate for infected target cells.

FIG. 6 presents a graph of initial viral load as a function of the specific death rate for infected target cells.

FIG. 7 presents a graph of critical efficacy as a function of the specific death rate for infected target cells.

FIG. 8 presents a graph of change in viral load as a function of time.

FIG. 9 presents a model-predicted viral kinetic response to various levels of induction therapy.

FIG. 10 presents a model-predicted viral kinetic response to various levels of treatment according to embodiments of the methods disclosed herein.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. As appropriate, procedures involving the use of commercially available kits and reagents are generally carried out in accordance with manufacturer defined protocols and/or parameters unless otherwise noted.

### DEFINITIONS

The term “administer” means to introduce a therapeutic agent into the body of a patient in need thereof to treat a disease or condition.

The term “continuous infusion system” refers to a device for continuously administering a fluid to a patient parenterally for an extended period of time or for intermittently administering a fluid to a patient parenterally over an extended period of time without having to establish a new site of administration each time the fluid is administered. The fluid typically contains a therapeutic agent or agents. The device typically has one or more reservoir(s) for storing the fluid(s) before it is infused, a pump, a catheter, cannula, or other tubing for connecting the reservoir to the administration site via the pump, and control elements to regulate the pump. The device may be constructed for implantation, usually subcutaneously. In such a case, the reservoir will usually be adapted for percutaneous refilling.

The term “treating” and/or “treatment” refers to the management and care of a patient having a pathology such as a viral infection or other condition for which administration of one or more therapeutic compounds is indicated for the purpose of combating or alleviating symptoms and complications of those conditions. Treating includes administering one or more formulations of the present invention to prevent the onset of the symptoms or complications, alleviating the symptoms or complications, or eliminating the disease, condition, or disorder. As used herein, “treatment” or “therapy” refer to both therapeutic treatment and prophylactic or preventative measures. In addition, “treating” or “treatment” does not require complete alleviation of signs or symptoms, does not require a cure, and specifically includes protocols which have only a marginal effect on the patient.

The term “therapeutically effective amount” refers to an amount of an agent (e.g. a cytokine such as interferon-α or small molecule inhibitors such as ribavirin) effective to treat at least one sign or symptom of a disease or disorder in a human. Amounts of an agent for administration may vary based upon the desired activity, the diseased state of the patient being treated, the dosage form, method of administration, patient factors such as the patient's sex, weight and age, the underlying causes of the condition or disease to be treated, the route of administration and bioavailability, the persistence of the administered agent in the body, the formulation, and the potency of the agent. It is recognized that a therapeutically effective amount is provided in a broad range of concentrations. Such range can be determined based on in vitro and/or in vivo assays.

The term “profile” is used according to its art accepted meaning and refers to the collection of results of one or more analyses or examinations of: (1) the presence of; or (2) extent to which an observed phenomenon exhibits various characteristics. Illustrative profiles typically include the results from a series of observations which, in combination, offer information on factors such as, for example, the presence and/or levels and/or characteristics of one or more agents infecting a patient (e.g. the hepatitis C virus), as well as the pharmacokinetic and/or pharmacodynamic characteristics of one or more therapeutic agents administered to a patient as part of a treatment regimen (e.g. interferon-α), as well as the physiological status or functional capacity of one or more organs or organ systems in a patient (e.g. the liver) etc.

The term “therapeutic regimen” is used according to its art accepted meaning and refers to, for example, a treatment plan for an individual suffering from a pathological condition (e.g. chronic hepatitis C infection) that specifies factors such as the agent or agents to be administered to the patient, the dosages of such agent(s), the schedule and duration of the treatment etc.

The term “pharmacokinetics” is used according to its art accepted meaning and refers to the study of the action of drugs in the body, for example the effect and duration of drug action, the rate at they are absorbed, distributed, metabolized, and eliminated by the body etc. (e.g. the study of a concentration of interferon-α in the serum of the patient that results from its administration via a therapeutic regimen). The term “pharmacodynamics” is used according to its art accepted meaning and refers to study of the biochemical and physiological effects of drugs on the body or on microorganisms or parasites within or on the body, the mechanisms of drug action and the relationship between drug concentration and effect etc. (e.g. the study of a concentration of hepatitis C virus RNA present in a patient's plasma following one or more therapeutic regimens).

The terms “continuous administration” and “continuous infusion” are used interchangeably herein and mean maintaining a minimal steady state serum level of an agent such as interferon throughout the course of the treatment period. This can be accomplished by constantly or repeatedly injecting substantially identical amounts of interferon (typically with a continuous infusion pump device), e.g., at least every hour, 24 hours a day, seven days a week, such that a steady state serum level is achieved for the duration of treatment. Continuous interferon may be administered according art accepted methods, for example via subcutaneous or intravenous injection at appropriate intervals, e.g. at least hourly, for an appropriate period of time in an amount which will facilitate or promote in vivo inactivation of hepatitis C virus.

The term “patients or humans having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections. These patients having chronic hepatitis C include those who are infected with multiple HCV genotypes including type 1 as well as those infected with, inter alia, HCV genotype 2 and/or 3. The term “pediatric patient” as used herein means a patient below the age of 17, and normally includes those from birth to 16 years of age. The term “treatment-naive patients having hepatitis C infections” as used herein means patients with hepatitis C who have never been treated with ribavirin or any interferon, including but not limited to interferon-alpha, or pegylated interferon alpha. The term “treatment-experienced patients having hepatitis C infections” as used herein means patients with hepatitis C who have been treated with ribavirin or any interferon, including but not limited to interferon-alpha, or pegylated interferon alpha, including relapsers and non-responders. The term “patients having chronic hepatitis C infections” as used herein means any patient having chronic hepatitis C and includes “treatment-naive patients” and “treatment-experienced patients” having chronic hepatitis C infections, including but not limited to relapsers and non-responders. The term “relapsers” as used herein means treatment-experienced patients with hepatitis C who have relapsed after initial response to previous treatment with interferon alone, or in combination with ribavirin. The term “non-responders” as used herein means treatment-experienced patients with hepatitis C who have not responded to prior treatment with any interferon alone, or in combination with ribavirin.

The term “wild-type Hepatitis C virus” is used herein according to its art accepted meaning and refers to the predominant genotypes of HCV that are found in nature, in contrast to induced mutations (e.g. those mutant forms of HCV that are observed to be induced upon exposure to a small molecule such as ribavirin), or mutations generated via some other form of genetic manipulation (see, e.g. MacParland et al., Journal of General Virology (2006), 87, 3577-3586 and Kieffer et al., Hepatology, 2007, 46(3): 631-639, the contents of which are incorporated herein by reference).

The term “cytokine” is a generic term for a class of polypeptides released by cells that act as mediators of a wide variety of physiological processes. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KL). The term “interferon” as used herein means the family of highly homologous species-specific proteins that inhibit viral replication and cellular proliferation and modulate immune response. Human interferons are grouped into three classes based on their cellular origin and antigenicity: α-interferon (leukocytes), β-interferon (fibroblasts) and γ-interferon (T cells). Recombinant forms of each group have been developed and are commercially available. Subtypes in each group are based on antigenic/structural characteristics. A number of α-interferons (grouped into subtypes) having distinct amino acid sequences have been identified by isolating and sequencing DNA encoding these peptides. Both naturally occurring and recombinant α-interferons may be used in the practice of the invention. As used herein, the term cytokine includes proteins from natural sources or from recombinant cell culture and biologically active equivalents of the native sequence cytokines.

The term “antibody” when used for example in reference to an “antibody capable of binding HCV” is used in the broadest sense and specifically covers intact monoclonal antibodies, polyclonal antibodies, multispecific antibodies (e.g. bispecific antibodies) formed from at least two intact antibodies, and antibody fragments so long as they retain their ability to immunospecifically recognize a target polypeptide.

### Illustrative Embodiments of the Invention

The invention disclosed herein has a number of embodiments. Typical embodiments of the invention include methods for obtaining patient-specific regimen responsiveness profiles based upon individualized patient factors such as infection parameters (e.g. hepatitis C viral load) and therapeutic agent responsiveness parameters (e.g. in vivo concentrations of interferon-α administered to the patient) and then using the regimen responsiveness profiles to design optimized therapeutic regimens for patients suffering from pathological conditions (e.g. Hepatitis C infections). In particular embodiments, such methods comprise determining patient-specific pharmacokinetic (pK) and pharmacodynamic (pD) parameters and then utilizing these parameters to design new therapeutic regimens. This is typically achieved by adjusting the therapeutic agent(s) used, by adjusting the rate or duration of therapeutic agent administration, or by adjusting the pK or pD model parameters. In certain embodiments, the invention provides a computer implemented system for: (1) constructing a patient-specific regimen responsiveness profiles and/or (2) delivering therapeutic agent(s) using optimized therapeutic regimens designed in response to such profiles.

One illustrative embodiment of the invention is a method of using a patient-specific regimen responsiveness profile obtained from a patient infected with hepatitis C virus (HCV) to design a patient-specific therapeutic regimen. This method comprises administering at least one therapeutic agent to the patient following a first therapeutic regimen and then obtaining pharmacokinetic or pharmacodynamic parameters from the patient in order to observe a patient-specific response to the first therapeutic regimen. Typically, pharmacokinetic or pharmacodynamic parameters observed comprise a concentration of the therapeutic agent in the blood of the patient that results from the first therapeutic regimen and/or a concentration of hepatitis C virus present in the patient. In this embodiment of the invention, practitioners can then use the pharmacokinetic or pharmacodynamic parameters observed in the patient in response to the first therapeutic regimen to obtain a patient-specific regimen responsiveness profile. This patient-specific regimen responsiveness profile is based upon an HCV infected patient's individualized physiology and necessarily takes into account a variety of host factors such as ethnicity, obesity, insulin resistance, hepatic fibrosis as well as viral factors such as genotype and baseline viral load. This patient-specific regimen responsiveness profile is then used to design a patient-specific therapeutic regimen.

By using the disclosed methods to obtain patient-specific regimen responsiveness profiles which are in turn used to design a patient-specific therapeutic regimens, practitioners can reduce or avoid complications in therapy that result from individualized factors such as ethnicity, obesity, insulin resistance, hepatic fibrosis as well as viral factors such as genotype and baseline viral load. As is disclosed in detail below, general embodiments of the invention are designed to overcome the general problems with individual patient factors (e.g. clinical cure rate for HCV of only about 50%). In addition, certain embodiments of the invention are tailored to address specific problems caused by individual patient factors (e.g. hepatic status and its associated influence on the serum plasma levels of interferon-α administered to the patient, the severity of side effects caused by interferon-α in that specific patient etc. etc.).

Embodiments of the invention can use information obtained from patient-specific regimen responsiveness profiles to design a variety of patient-specific therapeutic regimens. In typical embodiments of the invention, the first therapeutic regimen comprises interferon-α and the patient-specific therapeutic regimen is selected to modulate serum interferon-α concentrations in the patient. In other embodiments of the invention, the patient-specific therapeutic regimen is selected to maintain serum interferon-α concentrations in the patient at a value greater than a EC₅₀, a concentration at which the effectiveness of interferon-α is 50% of its maximum. In other embodiments of the invention, the patient-specific therapeutic regimen is selected to maintain serum interferon-α concentrations in the patient at a value where the actual efficacy of interferon-α in the patient is greater than the critical efficacy of interferon-α. In other embodiments, the patient-specific therapeutic regimen is selected to modulate interferon-α concentrations in the patient so that the patient is administered different interferon dosing regimens during different phases of hepatitis C viral load decline. In other embodiments, the patient-specific therapeutic regimen is selected to modulate interferon-α concentrations in the patient so that a difference between the actual efficacy of interferon-α and the critical efficacy of interferon-α in the patient is increased. In other embodiments, the patient-specific therapeutic regimen is selected to modulate interferon-α concentrations in the patient so as to reduce dose-dependent side effects observed during the administration of interferon-α. In certain embodiments of the invention, the patient-specific therapeutic regimen is designed to maintain plasma interferon-α levels in the patient above a set-point, e.g. above 10, 20, 30, 40, 50, 60, 70, 80, 90, 100, 110 or 120 IU/mL. In other embodiments of the invention, the patient-specific therapeutic regimen is designed to maintain plasma interferon-α levels in the patient below a set-point, e.g. below 140, 130, 120, 110, 100, 90, 80, 70 or 60 IU/mL (e.g. to reduce or avoid side-effects while maintaining a desired level efficacy).

In certain embodiments of the invention, measurements of phenomena such as the in vivo levels of an administered agent, the in vivo levels of HCV, the actual efficacy and limits of critical efficacy of such agents and the like are determined. Optionally, such determinations are made 0, 1, 2, 3, 4, 6, or 7 days (i.e. week 1) after the administration of a therapeutic regimen and/or any day of weeks 2, 3, 4, 5, 6, 7, 8, 9, 10, 11, 12 etc. up to for example week 72. In one illustrative embodiment, after the initiation of a therapeutic regimen, patients can return for safety and efficacy evaluations on a weekly basis up to week 4 and every 28 days thereafter throughout a 48 week treatment duration, with weekly or monthly follow-up visits up to week 72. Optionally determinations of actual efficacy and limits of critical efficacy occur between 0 and 7 days, and more preferably around between about 0 to 2 days. Alternatively, this determination may be made intermittently throughout therapy, to take into account for example individualized patient response to various therapeutic regimens. One with ordinary skill in the art will undoubtedly realize that different pharmacokinetic, pharmacodynamic, and viral kinetic models such as those described herein may be used to achieve this.

Certain embodiments of the invention use algorithms to obtain pharmacokinetic or pharmacodynamic parameters that comprise the patient-specific profile. Such embodiments of the invention can use one of a variety of art accepted methodologies. For example, certain embodiments of the invention can employ numerical methods with these equations and parameters. Some embodiments of the invention use analytical solutions to observe such parameters. In one illustrative embodiment of the invention, the values of such model parameters can be determined using standard non-linear regression techniques (see, e.g. Motulsky and Christopoulos “Fitting Models to Biological Data Using Linear and Nonlinear Regression: A Practical Guide to Curve Fitting” Oxford University Press, USA; 1 edition (May 27, 2004)). Those of skill in the art understand that there are many variations of these illustrative model equations that can be used in embodiments of the invention (e.g. those having small changes such as including time delays and the like) and that such variations are contemplated and encompassed by the disclosure provided herein.

In one embodiment of the invention that uses algorithms, the first therapeutic regimen comprises interferon-α and observed parameters comprise a concentration of administered interferon-α in the serum of the patient that is obtained from the patient using an algorithm comprising:

$$\frac{d D}{d t} = Q - {k}_{a} D$$

$$\frac{d C}{d t} = ( \frac{{k}_{a}}{{V}_{d}^{′}} ) D - {k}_{e} C$$

$$or$$

$$ɛ ( t ) = \frac{{C ( t )}^{n}}{{EC}_{50}^{n} + {C ( t )}^{n}}$$

wherein:

D represent dose of interferon in the infusion site (IU);

Q represents infusion rate of interferon (IU/hour);

kₐ represent interferon absorption rate constant (1/hour);

kₑ represents interferon elimination rate constant (1/hour);

Vd′ represents apparent volume of distribution (mL);

C represents plasma concentration of interferon (IU/mL);

EC₅₀ represents concentration at which drug's efficacy is half its maximum (IU/mL);

n represents Hill's coefficient; and

ε represents actual efficacy.

The variable and terms found in the various algorithms disclosed herein are used according to their art accepted definitions. See, e.g. Powers, et al. (2003). “Modeling viral and drug kinetics: hepatitis C virus treatment with pegylated interferon alfa-2b.” Semin Liver Dis 23 Suppl 1: 13-18 and Perelson, et al. (2005). “New kinetic models for the hepatitis C virus.” Hepatology 42(4): 749-754.

In some embodiments of the invention, observed parameters comprise a concentration of hepatitis C virus in one or more in vivo compartments (e.g. the viral load) and are obtained from the patient using an algorithm comprising:

$$\frac{d T}{d t} = s + rT ( 1 - \frac{T + I}{{T}_{max}} ) - dT - β VT$$

$$\frac{d I}{d t} = β VT + rI ( 1 - \frac{T + I}{{T}_{max}} ) - δ I$$

$$\frac{d V}{d t} = ( 1 - ɛ ) pI - cV$$

wherein:

T represents the concentration (density) of uninfected target cells (cells/ml);

I represents the concentration (density) of infected target cells (cells/ml);

Tₘₐₓ represents a maximum size of the liver (cells/ml)

V represents viral load (IU/ml);

s represents a constant rate of uninfected target cells production (cell ml⁻¹*day⁻¹);

r represents maximum specific proliferation rate of infected and uninfected target cells (day⁻¹);

βrepresents the infection rate constant rate (ml*day⁻¹*IU⁻¹);

p represents virion production rate constant (IU*cell⁻¹*day⁻¹);

c represents virion clearance rate constant (day⁻¹);

δ represents the specific death for infected target cells (day⁻¹);

d represents the specific death rate for uninfected target cells (day⁻¹); and

ε represents overall drug efficacy.

In some embodiments of the invention, observed parameters comprise the actual efficacy of the first therapeutic regimen and are determined using an algorithm comprising:

V(t)=V_{bar}[1−ε+εe^{-ct}]

### Wherein:

V(t) represents viral load (IU/ml);

V_{bar} represents initial viral load (IU/ml);

ε represents actual efficacy;

t represents time (day); and

c represents clearance constant (day⁻¹).

In some embodiments of the invention, observed parameters comprise the critical efficacy of the therapeutic agent in the first therapeutic regimen is determined in the patient is calculated using an algorithm comprising:

$${ɛ}_{c} = 1 - \frac{c ( δ {T}_{max} + r {\overline{T}}_{0} - {rT}_{max} )}{p β {T}_{max} {\overline{T}}_{0}}$$

wherein T̅ₒ is a number of uninfected target cells at uninfected steady state (I=V=0) which may be represented as:

$${\overline{T}}_{0} = \frac{{T}_{max}}{2 r} [ r - d + \sqrt{{( r - d )}^{2} + \frac{4 rs}{{T}_{max}}} ]$$

wherein r>d and s≦dTₘₐₓ so T̅ₒ≦T_{max.}

Those of skill in the art understand that factors such as epsilon can be determined in a variety of ways. Epsilon can be determined for example using equations 1-3 above in combination with observations of parameter values. Alternatively, epsilon can be determined for example using a constellation of PK/PD equations in combination with observations of C vs. t and V vs. t data.

The patient-specific profiles of the invention can be used to design patient specific therapeutic regimens in a number of contexts. For example, in embodiments of the invention where the profile includes assessments of HCV infected cells, the patient-specific therapeutic regimen can initiated when ratio of the number of HCV uninfected target cells to the number of HCV infected cells is greater than or equal to 1. In embodiments of the invention where the profile includes an assessment of plasma interferon-α concentrations, the patient-specific therapeutic regimen comprises administering a dose of interferon-α for a period of time selected to maintain a plasma interferon-α concentration above a set-point for the period of time. In certain embodiments of the invention, the patient-specific profile can be used to assess the patient's likely virological response to a defined interferon-α composition or a defined interferon-α dosing regimen. Those of skill in the art understand that a variety of patient-specific factors can be observed as part of a profile. For example, certain embodiments of the invention include observing additional patient-specific factors to those discussed above (e.g. “surrogate” PK markers such as PD marker molecules that are altered in response to the administration of interferon-α). Embodiments of the invention can examine for example, levels of beta-2-microglobulin, levels of neopterin, levels of 2′,5′ oligo-adenylate synthetase in a patient as well as the other markers disclosed herein and/or known in the art. For example, embodiments of the invention can also examine, a level of alanine transaminase or aspartate transaminase in plasma of the patient; a genotype or quasispecies of the hepatitis C virus; a patient's prior medical treatment history; and/or a presence or degree of a side effect that results from the first therapeutic regimen. In an illustrative embodiment where the therapeutic agent comprises interferon-α, one can also observe a presence or degree of a depression, a neutropenia, a thrombocytopenia, as well as one or more systemic flu-like symptoms that results from its administration.

Artisans have variety of methodologies for measuring markers observed in embodiments of the invention. Methods and materials used in the measurement of neopterin are described for example in Fernandez et al., J Clin Gastroenterol. 2000 30(2):181-6). Methods and materials used in the measurement of 2′,5′ oligo-adenylate synthetase are described for example in Podevin et al., J. Hepatol. 1997 (2):265-71). Methods and materials used in the measurement of beta-2-microglobulin are described for example in Malaquarnera Eur J Gastroenterol Hepatol. 2000 August; 12(8):937-9. Methods and materials used in the measurement of neutropenia and thrombocytopenia are described for example in Koskinas et al., Med. Virol. 2009 Mar. 24; 81(5):848-852. Methods and materials used in the measurement of neutropenia are described for example in Koskinas et al., Med. Virol. 2009 Mar. 24; 81(5):848-852. Methods and materials used in the measurement of alanine transaminase and/or aspartate transaminase are described for example in Sterling et al., Dig Dis Sci. 2008 May; 53(5):1375-82 Epub 2007. Methods and materials used in the measurement of depression (e.g. the Beck Depression Inventory) are described for example in Golub et al., J Urban Health. 2004 June; 81(2):278-90.

A wide variety of patient-specific therapeutic regimens can be designed using the patient-specific regimen responsiveness profiles disclosed herein. In typical embodiments of the invention, the patient-specific therapeutic regimen comprises administering interferon-α using a continuous infusion pump. Optionally the therapeutic regimen comprises an additional anti-viral agent such as ribavirin, VX-950; SCH 503034; R1626; or R71278. In certain embodiment of the invention, the patient-specific therapeutic regimen comprises administering a first dose of interferon-α (and/or ribavirin) during a first phase of hepatitis C viral decline and a second dose of interferon-α (and/or ribavirin) during a second phase of hepatitis C viral decline.

Once a first patient-specific regimen is designed and administered, practitioners can then obtain a further patient-specific regimen responsiveness profile that results from the administration first patient-specific regimen. Such further patient-specific regimen responsiveness profile can then be used to design further patient-specific regimens. For example, certain embodiments of the invention comprise obtaining pharmacokinetic or pharmacodynamic parameters from the patient so as to observe a patient-specific response to the first patient-specific therapeutic regimen as discussed above, wherein the pharmacokinetic or pharmacodynamic parameters comprise at least one of: a concentration of administered interferon-α in the plasma of the patient; or a concentration of hepatitis C virus in the plasma of the patient; using the pharmacokinetic or pharmacodynamic parameters observed in the patient in response to the first patient-specific therapeutic regimen to obtain a second patient-specific regimen responsiveness profile; and using the second patient-specific regimen responsiveness profile to design a second (or third or fourth etc.) patient-specific therapeutic regimen.

Certain embodiments of the invention can be implemented on a computer system. As discussed in detail below, embodiments of the invention are performed using computer systems. Typically, the systems include a controller programmed with mathematical models representing a viral response in a patient receiving a therapeutic regimen and programmed to regulate the dosing rate of therapeutic agent based on the models and the measurements of clinical parameters (e.g. in vivo concentrations of an administered therapeutic agent or viral load).

One such embodiment of the invention is a method of administering interferon-α to a patient suffering from a Hepatitis C infection, the method comprising: administering interferon-α to the patient following a first therapeutic regimen; obtaining pharmacokinetic or pharmacodynamic parameters from the patient to observe a patient-specific response to the first therapeutic regimen wherein the pharmacokinetic or pharmacodynamic parameters comprise at least one of: a concentration of interferon-α in the blood of the patient that results from the first therapeutic regimen; or a concentration of hepatitis C virus present in the patient. The pharmacokinetic or pharmacodynamic parameters so observed in the patient in response to the first therapeutic regimen are then used to design a patient-specific therapeutic regimen; one which can, for example, be programmed into a controller that operably coupled to a continuous infusion pump. The continuous infusion pump having this program can then be used to administer interferon-α to the patient according to the controller programming.

In one such computer implemented embodiment of the invention, the controller is programmed so that the continuous infusion pump administers interferon-α in a manner that: maintains serum interferon-α concentrations in the patient at a value greater than a EC₅₀, a concentration at which the effectiveness of interferon-α is 50% of its maximum; maintains serum interferon-α concentrations in the patient at a value where the actual efficacy of interferon-α in the patient is greater than the critical efficacy of interferon-α; modulates interferon-α concentrations in the patient so that the patient is administered different interferon dosing regimens during different phases of hepatitis C viral load decline; modulates interferon-α concentrations in the patient so that a difference between the actual efficacy of interferon-α and the critical efficacy of interferon-α in the patient is increased; or modulates interferon-α concentrations in the patient so as to reduce adverse side effects observed during the administration of interferon-α. In another computer implemented embodiment of the invention, the controller is programmed so that the continuous infusion pump administers interferon-α in a manner that: maintains serum interferon-α concentrations in the patient at a value less than a EC₅₀, a concentration at which the effectiveness of interferon-α is 50% of its maximum

In another computer implemented embodiment of the invention, the controller is programmed so that the continuous infusion pump administers interferon-α at a dose and for a period of time selected to maintain a plasma interferon-α concentration above a set-point for the period of time; and the patient-specific therapeutic regimen further comprises administering a nucleoside analog that interferes with Hepatitis C viral replication (e.g. ribavirin).

Another related embodiment of the invention is a system for administering interferon to a patient having a hepatitis C infection, the system comprising: a continuous infusion pump having a medication reservoir comprising interferon-α; and a processor operably connected to the continuous infusion pump that comprises a set of instructions that causes the continuous infusion pump to administer the interferon-α to the patient according to a patient-specific therapeutic regimen made by administering interferon-α to the patient following a first therapeutic regimen; obtaining pharmacokinetic or pharmacodynamic parameters from the patient so as to observe a patient-specific response to the first therapeutic regimen wherein the pharmacokinetic or pharmacodynamic parameters comprise at least one of: a concentration of interferon-α in the blood of the patient that results from the first therapeutic regimen; or a concentration of hepatitis C virus present in the patient; using the pharmacokinetic or pharmacodynamic parameters observed in the patient in response to the first therapeutic regimen to obtain a patient-specific regimen responsiveness profile; and then using the patient-specific regimen responsiveness profile to make the patient-specific therapeutic regimen. In certain embodiment of this system, the continuous infusion pump has dimensions smaller than 15×15 centimeters; and/or is operably coupled to an interface that facilitates the patient's movements while using the continuous infusion pump, wherein the interface comprises a clip, a strap, a clamp or a tape. In certain embodiment of this system the interferon-α delivered by this continuous infusion pump is not conjugated to a polyol.

Yet another embodiment of the invention is a program code storage device, comprising: a computer-readable medium; a computer-readable program code, stored on the computer-readable medium, the computer-readable program code having instructions, which when executed cause a controller operably coupled to a medication infusion pump to administer the interferon-α to a patient infected with the hepatitis C virus according to a patient-specific therapeutic regimen made by: administering interferon-α to the patient following a first therapeutic regimen obtaining pharmacokinetic or pharmacodynamic parameters from the patient so as to observe a patient-specific response to the first therapeutic regimen wherein the pharmacokinetic or pharmacodynamic parameters comprise at least one of: a concentration of interferon-α in the blood of the patient that results from the first therapeutic regimen; or a concentration of hepatitis C virus present in the patient; using the pharmacokinetic or pharmacodynamic parameters observed in the patient in response to the first therapeutic regimen to obtain a patient-specific regimen responsiveness profile; and then using the patient-specific regimen responsiveness profile to make the patient-specific therapeutic regimen.

The methods of the invention can be practiced on a wide variety of individuals infected with HCV including those previously treated for HCV infection or having a specific HCV strain. For example, some embodiments of the invention include the step of selecting the patient for treatment by identifying them as one previously treated with a course of interferon-α therapy, wherein the previous course interferon-α therapy was observed to be ineffective to treat one or more symptoms associated with the HCV infection. Other embodiments of the invention include the step of selecting the patient for treatment by identifying the patient as one infected with a specific HCV genotype, for example one infected with Genotype 1 or Genotype 1a.

In certain embodiments of the invention, the status of HCV in the individual is monitored during one or more of the phases of the viral life cycle. In particular, during chronic HCV infection, the level of serum HCV RNA does not vary significantly (<0.5 log) on time scales of weeks to months. However, when patients chronically infected with HCV are treated with interferon-α (IFN) or IFN plus ribavirin, HCV RNA generally declines after a 7-10 hour delay. The typical decline is biphasic and consists of a rapid first phase lasting for approximately 1-2 days during which HCV RNA, on average, may fall 1 to 2 logs in genotype 1 infected patients and as much as 3 to 4 logs in genotype 2 infected patients. Subsequently, a slower second phase of HCV RNA decline ensues. Triphasic viral declines also have been observed in some patients. A triphasic decline consists of a first phase (1-2 days) with rapid virus load decline followed by a shoulder phase (4-28 days)—in which virus load decays slowly or remains constant—and a third phase of renewed viral decay. In nonresponders, there may be no viral decline (null response) or a first phase followed by no second-phase decline (flat partial response) or rebound to baseline level.

In certain embodiments of the invention, the status of HCV in the individual is monitored during one or more of the phases of the viral life cycle so as to obtain information useful in the tailoring of the therapeutic regimen to the viral phase in a specific individual. Typically, in certain embodiments of the invention, the initial and then changing concentrations of hepatitis C virus in the serum of the patient can be measured by a quantitative PCR method that is employed during the various phases of the viral decline that occurs in response to one or more therapeutic regimens. In one illustrative embodiment, the status of HCV in the individual is monitored over a period of time so as to determine if one or more therapeutic regimens is sufficient to reduce the levels of hepatitis C virus at least 1, 2, 3, 4, 5 or 6 logs. In another illustrative embodiment, the status of HCV in the individual is monitored over a period of time so as to determine if a therapeutic regimen is sufficient to reduce the concentration of hepatitis C virus to below the detection limit of the assay (typically 10-100 IU/mL of serum or plasma; e.g. during the first, second or third phases and/or at the junctions between these different phases of hepatitis C viral decline). In the embodiments of the invention that examine viral load, those of skill in the art understand that units of viral load, which are expressed a number of ways in the literature including: (1) IU/mL—international units/mL; (2) (RNA) copies/mL; and (3) virions/mL (see, e.g. Saldanha et al., Vox Sang 1999; 76:149-158). Those of skill in the art further understand that the IU-s used to characterize HCV levels are different from the IU-s used to characterize interferon-α levels.

In some embodiments, interferon may be administered at a first dosing rate during the first stage and a second dosing rate during the second stage, higher than the first dosing rate, i.e. or resulting in higher efficacy than the first dosing rate, followed by a dosing rate calculated to result in efficacy determined by fitting the viral model. By way of non-limiting example, the first stage may last between about 1 and 12 weeks, more preferably between about 3 to 5 weeks, and more preferably for about 4 weeks. The second stage may last for about 2 to 4 weeks. Finally, for the remainder of the therapy, the patient may be administered interferon at a dosing rate adjusted based on patient's actual and critical efficacy as described above. In one specific embodiment, the first dosing rate may be set to about 3 to 9 MIU/day (based on a 75 kg patient), preferably 6 MIU/day, and the dosing rate during the second stage may be set to about 9 MIU/day to 20 MIU/day, preferably to 12 MIU/day/75-kg patient. Alternatively, interferon may be administered at a dosing rate calculated to result in higher efficacy or maximized difference between actual efficacy and critical efficacy first. The first stage may then be followed by a stage with lower efficacy, by a stage where efficacy is calculated as described above, or both.

Typical interferons for use in embodiments of the invention include interferon α-2b (Intron A) (which is not pegylated) and pegylated interferon α-2b (PegIntron, PEG-IFN). Embodiments of the invention can include doses of Intron A that rage from about 3, 6, 9 or 12 million IU/day, and/or PegIntron 1.5 μg/kg once weekly via SC injection and/or continuous SC Intron A (80,000, 120,000, or 160,000 IU/kg/day) in a 1:1:1:1 ratio. Continuous SC delivery of Intron A can be achieved via the Medtronic MiniMed Paradigm infusion system for 24, 26, 48 60, 72 etc. weeks of therapy. Typically, patients will also receive 1000-1600 mg/day oral ribavirin by mouth daily based upon weight (e.g. 1000 mg/day if weight ≦75 kg; 1200 mg/day if weight >75 kg etc.). Individuals in such studies can include those with HCV genotype 1 infection who have had no previous interferon treatment, or alternatively HCV genotype 1 or 4 infection non-responders (e.g. individuals who have had previous interferon treatment but relapsed etc.).

In embodiments of the invention, a patient's responses to various therapeutic regimens administered according to embodiments of the invention can be examined by a variety of methods known in the art. Typical efficacy variables can be assessed in response to an HCV infected patient's treatment regimen and can include for example assessments of rapid virologic response (RVR): Undetectable HCV RNA level in response to a certain therapeutic regimen; as well as early virologic response (EVR): ≧2-log₁₀ reduction in HCV RNA level in response to a certain therapeutic regimen as compared with the baseline level etc.

Further illustrative methods and materials useful in practicing embodiments of the invention are discussed in detail below.

### Illustrative Methods and Materials for Observing HCV in Embodiments of the Invention

Hepatitis C virus (HCV) is a positively stranded RNA virus that exists in at least six genetically distinct genotypes. These genotypes are designated Type 1, 2, 3, 4, 5 and 6, and their full length genomes have been reported (see, e.g. Genbank/EMBL accession numbers Type 1a: M62321, AF009606, AF011753, Type 1b: AF054250, D13558, L38318, U45476, D85516; Type 2b: D10988; Type 2c: D50409; Type 3a: AF046866; Type 3b: D49374; Type 4: WC-G6, WC-G11, WG29 (Li-Zhe Xu et al, J. Gen. Virol. 1994, 75: 2393-98), EG-21, EG-29, EG-33 (Simmonds et al, J. Gen. Virol. 1994, 74: 661-668), the contents of which are incorporated by reference). In addition, viruses in each genotype exist as differing “quasispecies” that exhibit minor genetic differences. The vast majority of infected individuals are infected with genotype 1, 2 or 3 HCV. HCV infection affects approximately 1.8% of the population in the USA and 3% of the population of the world. In over 85% of infected people, HCV causes a lifelong infection characterized by chronic hepatitis that varies in severity between individuals.

A person suffering from chronic hepatitis C infection may exhibit one or more of the following signs or symptoms which can be examined (typically in addition to other factors) in order to obtain a patient-specific profile: (a) elevated serum alanine aminotransferase (ALT), (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose hepatitis C, but can be used to evaluate a patient's response to drug treatment. Elevated serum ALT and aspartate aminotransferase (AST) are known to occur in uncontrolled hepatitis C, and a complete response to treatment is generally defined as the normalization of these serum enzymes, particularly ALT (Davis et al., 1989, New Eng. J. Med. 321:1501-1506). ALT is an enzyme released when liver cells are destroyed and is symptomatic of HCV infection. Interferon causes synthesis of the enzyme 2′,5′-oligoadenylate synthetase (2′5′OAS), which in turn, results in the degradation of the viral mRNA. Houglum, 1983, Clinical Pharmacology 2:20-28. Increases in serum levels of the 2′5′OAS coincide with decrease in ALT levels. Histological examination of liver biopsy samples may be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity, the contents of which are incorporated by reference.

As discussed in detail below, certain embodiments of the invention include the step of monitoring the HCV viral load in a subject and to adjust the therapeutic regimen based upon the observed result. Similarly, in certain embodiments of the invention, whether a particular method or methodological step (e.g. a specific regimen) is effective in combating an HCV infection can be determined by a number of factors, typically by measuring viral load. Alternatively, in certain circumstances, one can measure another parameter associated with HCV infection, including, but not limited to, liver fibrosis.

Viral load can be measured by a variety of procedures known in the art, for example, by measuring the titer or level of virus in serum. These methods include, but are not limited to, a quantitative polymerase chain reaction (PCR) and/or a branched DNA (bDNA) test. Many such assays are available commercially, including a quantitative reverse transcription PCR(RT-PCR) (Amplicor HCV Monitor™ Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Illustrative assays used in embodiments of the invention to monitor viral titer in the methods of the invention include the COBAS Hepatitis C Virus (HCV) TaqMan Analyte-Specific Reagent Assay and/or the COBAS Amplicor HCV Monitor V2.0 and/or the Versant HCV bDNA 3.0 Assays (see, e.g. Konnick et al., Journal of Clinical Microbiology, May 2005, p. 2133-2140, Vol. 43, No. 5, the contents of which are incorporated by reference).

In certain embodiments of the invention, and HCV infected individual is administered a therapeutic agent such as interferon and/or a small molecule inhibitor such as ribavirin and the response to such agents is then observed by monitoring changes in the levels of HCV-RNA that are detectable in vivo, for example HCV-RNA copy number per milliliter of blood. In this context, an appropriate therapeutic response is associated with decreasing levels of HCV-RNA that are detectable in the blood of an infected individual. Ideally, a therapeutic regimen will reduce this number so that there is no longer any detectable HCV-RNA.

The term “no detectable HCV-RNA” in the context of the present invention means that there are fewer than 100 and typically fewer than 50 copies of HCV-RNA per ml of serum of the patient as measured by quantitative, multi-cycle reverse transcriptase PCR methodology. HCV-RNA is typically measured in the present invention by research-based RT-PCR methodology well known to the skilled clinician. This methodology is referred to herein as HCV-RNA/qPCR. The lower limit of detection of HCV-RNA is typically 10-100 IU/mL. Serum HCV-RNA/qPCR testing and HCV genotype testing will be performed by a central laboratory. See also J. G. McHutchinson et al. (N. Engl. J. Med., 1998, 339:1485-1492), and G. L. Davis et al. (N. Engl. J. Med. 339:1493-1499, the contents of which are incorporated by reference).

While viral titers are the most important indicators of effectiveness of a dosing regimen, other parameters can also be measured as secondary indications of effectiveness. Secondary parameters include reduction of liver fibrosis; and reduction in serum levels of particular proteins. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20, the contents of which are incorporated by reference. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Another alternative but indirect method of determining viral load is by measuring the level of serum antibody to HCV. Methods of measuring serum antibody to HCV are standard in the art and include enzyme immunoassays, and recombinant immunoblot assays, both of which involve detection of antibody to HCV by contacting a serum sample with one or more HCV antigens, and detecting any antibody binding to the HCV antigens using an enzyme labeled secondary antibody (e.g., goat anti-human IgG). See, e.g., Weiss et al. (1995) Mayo Clin. Proc. 70:296-297; and Gretch (1997) Hepatology 26:43 S-47S, the contents of which are incorporated by reference.

Serum markers of liver fibrosis can also be measured as an indication of the efficacy of a subject treatment method (e.g. a first therapeutic regimen). Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase. Yet another secondary indicator of effectiveness of a treatment regimen is a change in the levels of serum alanine aminotransferase (ALT). Serum ALT levels are measured, using standard assays. In general, an ALT level of less than about 80, less than about 60, less than about 50, or about 40 international units per liter of serum is considered normal. In some embodiments, an effective amount of IFN-α is an amount effective to reduce ALT levels to less than about 200 IU, less than about 150 IU, less than about 125 IU, less than about 100 IU, less than about 90 IU, less than about 80 IU, less than about 60 IU, or less than about 40 IU.

### Illustrative Therapeutic Agents for Use in Embodiments of the Invention

Embodiments of the invention can use a wide variety of therapeutic agents known in the art to both construct patient-specific profiles and then deliver therapeutic agent(s) using optimized regimens based upon these profiles. Typical embodiments of the methods disclosed herein include the administration of interferon-α or “interferon-alpha” to an individual infected with HCV. Such embodiments of the invention optimize regimens for treating HCV infection using permutations of ribavirin and an interferon alpha treatments that are well known in the art, e.g., as disclosed in U.S. Pat. No. 6,299,872, U.S. Pat. No. 6,387,365, U.S. Pat. No. 6,172,046, U.S. Pat. No. 6,472,373, and U.S. Patent Application No. 200060257365, the disclosures of which are incorporated herein by reference. The term “interferon-alpha” as used herein means the family of highly homologous species-specific proteins that inhibit viral replication and cellular proliferation and modulate immune response. Typical suitable interferon-alphas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon alpha-2c such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Welicome Ltd., London, Great Britain, or a consensus alpha interferon such as those described in U.S. Pat. Nos. 4,897,471 and 4,695,623 and the specific product available from Amgen, Inc., Newbury Park, Calif., or interferon alfa-n3 a mixture of natural alpha interferons made by Interferon Sciences and available from the Purdue Frederick Co., Norwalk, Conn., under the Alferon Tradename or recombinant interferon alpha available from Frauenhoffer Institute, Germany or that is available from Green Cross, South Korea. The use of interferon alfa-2a or alpha 2b is typical. Since interferon alpha 2b, among all interferons, has the broadest approval throughout the world for treating chronic hepatitis C infection, it is most typical. The manufacture of interferon alpha 2b is described in U.S. Pat. No. 4,530,901, the contents of which are incorporated by reference.

Various interferons available on the market include, but are not limited to, IFN-α: Roferon®-A, Intron®-A; consensus IFN: Infergen®; IFN-βs: Betaseron®, Rebif®, Avonex®, Cinnovex® and Berlex. Pegylated interferon-alpha-2b was approved in January 2001 and pegylated interferon-alpha-2a was approved in October 2002. Examples of commercially available pegylated interferons include, but are not limited to, PEGASYS®, PegIntron™ and Reiferon Retard®. As is known in the art, different preparations of therapeutic molecules such as the interferons do not exhibit identical activities and such activities are therefore published by the manufacturer. For example, for Infergen, the published activity is 1×10⁹ U/mg or 1 MIU/ug. For Pegylated Interferon alpha 2b, (PegIntron) the published (package insert) is 0.7×10⁸ U/mg or 70,000 U/ug. For Pegylated interferon alpha 2a (Pegasys) the published data suggest that the pegylated product has 7% the activity of the non-pegylated product. In typical embodiments, bio-potent non-pegylated interferon-alpha (IFN-α-2a or IFN-α-2b) or consensus interferon is used.

Intron-a (interferon-α 2b, Schering Plough) was a first interferon approved for hepatitis C use. Intron-a is also indicated for a variety of cancer therapies including a list of hematological malignancies and hepatitis B. There is no mention of therapy failures in the Intron-a package insert, however the label for Intron-a plus ribavirin therapy is indicated only for naïve patients. The dosages for hepatitis C therapy are listed as 3 MU TIW followed by a 50% dose reduction if not tolerated well. Roferon (interferon-α 2a, Roche) is another interferon approved for hepatitis C. The indication list is almost identical and the dosage is the same, 3 MU TIW. There is no indication of use with ribavirin and no discussion of therapy failures in the package labeling. Infergen (interferon-α consensus, Valeant) is labeled only for hepatitis C. Infergen is labeled as a 9 μg injection TIW in naïve patients and 15 μg TIW for patients who tolerated interferon but did not respond or relapsed. Peg-Intron (interferon-α 2b pegylated with a 12 kD PEG (polyethylene glycol), Schering Plough) was the first pegylated interferon introduced to the marketplace. Pegylation of the interferon leads to a molecule with reduced biological activity but a greatly increased circulating half-life in-vivo. Peg-Intron is labeled for weight based dosing with a single weekly injection in combination with ribavirin. Peg-intron is only labeled for naïve patients. The half-life of Peg-Intron is about 48 hours, so plasma levels of interferon are essentially zero by the end of day 7 following injection. Pegasys (interferon-α 2a pegylated with a 40kD PEG, Roche) was the second pegylated interferon approved for clinical use. In contrast to Peg-Intron, Pegasys is typically delivered at the same dose for all patients; however the ribavirin component is typically dosed by weight Like Peg-Intron, Pegasys is only indicated for interferon naïve patients. The pharmacokinetics of Pegasys are considerably different than Peg-intron due to the larger molecular weight of the PEG attached to the interferon. The circulating half-life of Pegasys is about 3 weeks, which might have considerable safety implications in the case of overdosing but does not allow for significantly reduced trough levels in the plasma. It is interesting to note that in both controlled clinical trials and in community practice, Peg-Intron And Pegasys therapies lead to very similar outcomes.

As noted above, certain embodiments of the methods disclosed herein include the administration of interferon-α that is conjugated to a polyol such as polyethylene glycol. Such interferon-α conjugates can be prepared by coupling an interferon alpha to a variety of water-soluble polymers. A non-limiting list of such polymers include polyethylene and polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090, the contents of which are incorporated by reference. The typical polyethylene-glycol-interferon alfa-2b conjugate is PEG₁₂₀₀₀-interferon alpha 2b. The phrases “12,000 molecular weight polyethylene glycol conjugated interferon alpha” and “PEG₁₂₀₀₀-IFN alpha” as used herein mean conjugates such as are prepared according to the methods of International Application No. WO 95/13090 and containing urethane linkages between the interferon alfa-2a or -2b amino groups and polyethylene glycol having an average molecular weight of 12000.

In certain embodiments of the invention, an interferon-α administered in one or more of the sequential phases is not conjugated to a polyol. In some embodiments of the invention, the interferon-α so administered comprises two interferon-α species: a first interferon-α species that is conjugated to a polyol; and a second interferon-α species that is not conjugated to a polyol. Optionally different species of interferon-α are administered in one or more of the different sequential phases of the invention.

To minimize the number of pump refills during the therapy, the supply of interferon in the pump may last for an extended period of time. Because the loadable amount of interferon is fixed by the drug reservoir volume, to increase the amount of time the interferon supply may last, potency of interferon, as well as concentration of interferon may be increased. Accordingly, in some embodiments, the interferon may comprise a highly potent interferon. The term “highly potent” means an interferon that may exhibit favorable characteristics such as antiviral activity, antiproliferative activity, efficacy in clearing hepatitis virus from cells, increased ratio of antiviral activity to antiproliferative activity, or increased ratio of Tₕ1 differentiation activity to antiproliferative activity. Due to these characteristics, less volume of interferon is required to cause the same therapeutic effect on the patient, and thus highly potent interferon formulation may be administered at a lower flow rate. Alternatively, a highly soluble interferon may be used to prepare formulations with increased concentration of interferon, which can also be administered at a lower flow rate. The term “highly soluble” means interferon with a solubility of between about 5 mg/ml to about 10 mg/ml. In typical embodiments, the interferon concentration may be between about 360 MIU/ml to about 1500 MIU/ml.

In practicing the methods of the invention, the therapeutic regimen(s), e.g. the therapeutic agent(s), the dosage amount(s), dosage period(s), dosage schedule(s), dosage route(s), and so on, for agents such as interferon-α and/or ribavirin, encompass those generally used in the art to administer these agents in a manner that typically produces an improvement in one or more physiological conditions associated with a chronic hepatitis C infection. In this context, skilled artisans understand that a variety of therapeutic regimens known in the art can be employed in and/or adapted to the methods of the invention (e.g. those described in United States Patent Applications 2006/0088502 and 2006/0024271 and U.S. Pat. No. 6,849,254 the contents of which are incorporated by reference).

As is known in the art, interferon-α can be administered at a fairly high dose (e.g. up to 300 million IU/m² subcutaneously) without adverse reactions. In this context, medical personnel typically control and/or modify an interferon-α dosage regimen depending on the constellation of clinical factors observed in a specific individual (factors which are known to change during treatment). In particular, artisans understand that for HCV infections, one single predetermined regimen is not applicable to all patients and that optimally effective regimens are typically those that are individually designed in view of various factors observed in a specific individual. For example, medical personnel may select a specific interferon-α dosage regimen based upon the genotype or subtype of HCV that is observed to be infecting the patient and/or the amount of HCV-RNA per ml of serum in the patient as measured by a quantitative PCR method. As is similarly known in the art, the dosage regimen may be selected or controlled depending on the weight and age of a patient, whether the patient is known to be a nonresponder or relapser, or whether the patient is observed to have another pertinent pathological condition (e.g. cirrhosis of the liver, hepatocarcinoma, HIV infection, or the like). Depending upon, for example, the constellation clinical factors observed in a specific individual, the interferon-α (and ribavirin) can be administered on a weekly (QW), twice a week (BIW), three times a week (TIW), every other day (QOD) or on a daily basis. Similarly, depending upon for example the clinical factors and/or personal needs of the patients, these therapeutic agents can be administered via a variety of routes, for example subcutaneously, intramuscularly or intravenously. In certain dosage regimens, an infusion delivery device (e.g. a medication infusion pump) is used to deliver interferon-α. In other dosage regimens, an infusion delivery device is not used, and the interferon-α is delivered via injection with a conventional syringe. In this context, the following descriptions of various illustrative schemes for administering therapeutically effective amounts of the combination therapy of interferon-α and ribavirin are not limiting and are instead provided merely as typical examples of dosage regimens known in the art that can be employed and/or adapted to the methods of the invention.

In typical embodiments of the invention, the interferon-α administered is selected from one or more of interferon alpha-2a, interferon alpha-2b, a consensus interferon, a purified interferon alpha product (e.g. a purified interferon-α product produced by a recombinant technology) and/or a pegylated interferon-α. As is known in the art, an interferon-α dose can be characterized in international units (IU) or milligrams of polypeptide, optionally in the context of amount of agent per kilogram of patient weight and/or another measure of patient size (e.g. m²). In one illustrative embodiment of the invention, the interferon-α can be consensus interferon and the amount of interferon-α administered can be from 1 to 20 micrograms per week on a weekly (QW), twice a week (BIW), three times a week (TIW), every other day (QOD) or on a daily basis. Alternatively, the interferon-α administered can be a pegylated interferon alpha-2a and the amount of interferon-α administered is from 20 to 250 micrograms/kilogram per week on a weekly, TIW, QOD or daily basis. In another embodiment, the interferon-α administered can be a pegylated interferon alpha-2b and the amount of interferon-α administered can be from 0.5 to 2.0 micrograms per week on a QW, BIW, TIW, QOD, or on a daily basis. Optionally, the interferon-α can be selected from interferon alpha-2a, interferon alpha-2b, or a purified interferon-α product and the amount of interferon-α administered can be from 1 to 20 million IU per week on a daily to weekly basis. In one embodiment, the interferon-α administered is interferon-alpha-2b and the amount of interferon-α administered can be 2 to 10 (and typically 4, 5, 6, 7, or 8) million IU three to seven times a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe. While administration or infusion can be intermittent or continuous, the frequency of injection of the interferon composition will typically depend on the form of the composition. It will be understood that the injection will be less frequent (e.g., once or twice a week) when using sustained release formulations or long-acting polymer conjugates (e.g. those conjugated with polyethylene glycol).

In certain embodiments when the interferon-α administered is selected from interferon alfa-2a, interferon alfa-2b, or a purified interferon-α product, the therapeutically effective induction dosing amount of interferon-α administered in the induction and/or final phases can be 6-10 MIU daily for a first specific time period (e.g. 2 weeks), followed by 3-5 MIU daily for another time period (e.g. 6 weeks), followed by 1-3 MIU daily for yet another time period (e.g. 16 weeks to 24 weeks). When the interferon-α administered is consensus interferon, the amount of consensus interferon administered in the first treatment period of twenty-four weeks can be from, for example, 15 to 20 micrograms on a daily basis for two or more weeks, followed by 9 to 15 micrograms on a daily basis for twenty or more weeks. In such dosage regimes that are adapted to the methods of the invention, an infusion delivery device (e.g. a medication infusion pump) can be used to deliver interferon-α. Alternatively, an infusion delivery device is not used in such dosage regimes, and the interferon-α is delivered via injection with a conventional syringe.

In certain embodiments where the pegylated interferon-α is a pegylated interferon alfa-2b, the therapeutically effective amount of pegylated interferon alfa-2b administered during a phase of the treatment can be the range of about 0.1 to 9.0 micrograms per kilogram of pegylated interferon alfa-2b administered per week, in single or divided doses, for example once a week or twice a week, typically in the range of about 0.1 to about 9.0 micrograms per kilogram of pegylated interferon alfa-2b administered once a week or can be in the range of about 0.05 to about 4.5 micrograms per kilogram of pegylated interferon alfa-2b administered twice a week, or can be in the range of about 0.5 to about 3.0 micrograms per kilogram of pegylated interferon alfa-2b administered per week, for example in the range of about 0.5 to about 3.0 micrograms per kilogram of pegylated interferon alfa-2b administered once a week or in the range of about 0.25 to about 1.5 micrograms per kilogram of pegylated interferon alfa-2b administered twice a week, or can be in the range of about 0.75 to about 1.5 micrograms per kilogram of pegylated interferon alfa-2b administered per week, typically in the range of about 0.75 to about 1.5 micrograms per kilogram of pegylated interferon alfa-2b administered once a week or about 0.375 to about 0.75 micrograms per kilogram of pegylated interferon alfa-2b administered twice a week. When the pegylated interferon-α administered as part of the combination therapy is a pegylated interferon alfa-2a, the therapeutically effective amount of pegylated interferon alfa-2a administered during the treatment in accordance with the present invention can be in the range of about 50 micrograms to about 500 micrograms once a week, for example about 180 micrograms to about 250 micrograms QW or the effective amount is in the range of about 50 micrograms to about 250 micrograms twice a week, for example about 90 micrograms to about 125 micrograms twice a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In some embodiments where pegylated interferon-α is administered to pediatric patients as part of the methods of the invention is a pegylated interferon alfa-2b, the therapeutically effective amount of pegylated interferon alfa-2b administered during the treatment in accordance with the present invention can be in the range of about 0.1 to 9.0 micrograms per kilogram of pegylated interferon alfa-2b administered per week, in single or divided doses, optionally once a week or twice a week, typically about 0.1 to about 9.0 micrograms per kilogram of pegylated interferon alfa-2b administered once a week, or about 0.05 to about 4.5 micrograms per kilogram of pegylated interferon alfa-2b administered per week, in single or divided doses, optionally once a week or twice a week, for example from about 0.05 to about 4.5 micrograms per kilogram of pegylated interferon alfa-2b administered once a week, or optionally about 0.75 to about 3.0 micrograms per kilogram of pegylated interferon alfa-2b administered in single or divided doses, optionally once a week or twice a week, for example about 0.75 to about 3.0 micrograms per kilogram of pegylated interferon alfa-2b administered once a week or about 0.375 to about 1.5 micrograms per kilogram of pegylated interferon alfa-2b administered twice a week, and in certain embodiments about 2.25 to about 2.6 micrograms per kilogram of pegylated interferon alfa-2b administered once a week or about 1.1 to about 1.3 micrograms per kilogram of pegylated interferon alfa-2b administered twice a week. When the pegylated interferon-α administered to a pediatric patient is a pegylated interferon alfa-2a, the therapeutically effective amount of pegylated interferon alfa-2a administered during the treatment in accordance with the present invention can be in the range of about 50 micrograms to about 500 micrograms once a week, for example about 300 micrograms to about 375 micrograms QW or the therapeutically effective amount of pegylated interferon alfa-2a administered to a pediatric patient is in the range of about 50 micrograms to about 250 micrograms twice a week, optionally about 150 micrograms to about 190 micrograms once a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In certain embodiments of the invention, interferon-α is administered at 1-20 million IU/m², for example daily, either intravenously, intramuscularly, or subcutaneously. Treatments with interferon-α at this range of doses and route of administration can last for example from about two weeks to six months or a year. In some embodiments of the invention, 2 to 15 (and typically is 4, 5, 6, 7, 8 or 9) million IU a day of an interferon-α is subcutaneously, intramuscularly or intravenously administered in a single dose or in divided doses every day or intermittently, for instance 2, 3, 4, 5, 6 or 7 times a week, for a period of 2 to 48 weeks or longer. In certain embodiments for example, in one or more of the phases of the invention an amount such as 6 to 15 million IU of interferon-α a day is administered every day for a first defined period, for example 2 to 8 weeks and then intermittently for a second defined period, for example, 22 to 46 weeks. As is known in the art, such a regimen, however, may appropriately be changed depending on the kind or dosage form of interferon-α. In the case of PEGylated interferon-α 2a (Pegasys, manufactured by Chugai Pharmaceutical Co., Ltd.) for example, it is generally possible to subcutaneously administer the interferon once a week, every time at a dose of, for example 100-200 μg. In another illustrative embodiment of the invention, interferon-α is administered to the patient at 150 μg by subcutaneous injections. This treatment can typically last for four weeks or longer. In certain embodiments of the invention a recombinant interferon-α can be administered to a patient at doses of 0.01 to 2.5 mg/m² by intramuscular and/or intravenous bolus injections or alternating intramuscular and intravenous bolus injections with a minimum intervening period of 24, 48 or 72 hours. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

Since interferon may be exposed to elevated temperatures and/or mechanical stresses for an extended period of time, it may be desirable to prepare interferon compositions that enhance the stability of the interferon and prevent its degradation. In one embodiment, interferon may be stabilized in an aqueous medium by a mixed buffer system. For example, U.S. Pat. No. 6,734,162 discloses methods and materials that may be employed to prepare such compositions. Various other methods known and used in the art may also be used.

Because interferons may cause adverse side effects, in some embodiments, they may be delivered in a manner that provides increased levels of the drug in liver tissues and decreased levels in non-liver tissues. In one embodiment, it may be accomplished by chemically modifying the interferon to render it inactive until the modification is cleaved off by a liver-specific enzyme. One example of such technology, known as HepDirect, is offered by Metabasis Therapeutics, Inc, La Jolla, Calif. In another embodiment, the interferons may be modified to enhance its site-specific delivery to target cells. Suitable compounds for modifying the interferons in this manner include, but are not limited to, lactosaminated albumin, (Stefano, J. Pharmacol. Exp. Ther., May 2002; 301: 638-642) or galactosylated poly(L-lysine) (Gal-PLL) (Zhu et al., Bioconjugate Chem., 19 (1), 290-298, 2008). In yet other embodiments, interferon may be delivered via a drug delivery device either intraperitoneally or directly to the liver, slightly upstream from the liver vascular bed, such as into the hepatic artery.

In vivo samples (e.g. blood, serum, plasma, tissue etc.) may be assayed for interferon concentrations using a variety of different methods known and used in the art. One suitable example is an electrochemiluminescence-based assay and an ORIGEN analyzer (IGEN International, Inc. Gaithersburg, Md.). Other methods used in the art include those disclosed for example in Pirisi et al., Digestive Diseases and Sciences, 42(4): 767-7771 (1997); and Lam et al., Digestive Diseases and Sciences, 42(1):178-85 (1997).

In some embodiments of the invention, an interferon may be administered to a patient in combination with other antiviral agent(s). Combination therapy is particularly desirable for patients who suffer from an ongoing (chronic) hepatitis infection. Suitable anti-viral agents include, for example HCV polymerase or protease inhibitors. These anti-viral agents are typically administered orally.

Embodiments of the methods disclosed herein include the administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The in vitro inhibitory concentrations of ribavirin are disclosed in Goodman & Gilman's “The Pharmacological Basis of Therapeutics”, Ninth Edition, (1996) McGraw Hill, New York, at pages 1214-1215. The Virazole product information discloses a dose of 20 mg/mL of Virazole aerosol for 18 hours exposure in the 1999 Physicians Desk Reference at pages 1382-1384. Typical ribavirin dosage and dosage regimens are also disclosed by Sidwell, R. W., et al. Pharmacol. Ther 1979 Vol 6. pp 123-146 in section 2.2 pp 126-130. Fernandes, H., et al., Eur. J. Epidemiol., 1986, Vol 2(1) ppl-14 at pages 4-9 disclose dosage and dosage regimens for oral, parenteral and aerosol administration of ribavirin in various preclinical and clinical studies.

Suitable examples of ribavarin include, but are not limited to, Copegus®, Rebetol®, Ribasphere®, Vilona®, Virazole®, in addition to generic versions of the drug. Ribavirin is typically available in 200-mg capsules with the daily dosage calculated based on patient's weight or viral genotype. A person with ordinary skill in the art will undoubtedly be capable of determining the proper dosage to be administered. For example, for patient with viral genotype 1, the daily dosage may be 1,200 mg for patients that weigh over 165 lbs and 1,000 mg for patients that weigh less than 165 lbs. On the other hand, for patients with viral genotypes 2 or 3, the daily dosage may be set to about 800 mg regardless of the patient's weight. Suitable inhibitors include, but are not limited to, telapravir and others described below and in U.S. Pat. Nos. 5,371,017, 5,597,691, and 6,841,566.

Ribavirin is typically administered as part of a combination therapy to a patient in association with interferon-α, that is, before, after or concurrently with the administration of the interferon-α. The interferon-α dose is typically administered during the same period of time that the patient receives doses of ribavirin. The amount of ribavirin administered concurrently with the interferon-α typically varies depending upon various factors such as a patient's weight and can be less than 399 mg per day or from about 400 to about 1600 mg per day, e.g. 600 to about 1200 mg/day, or 800 to about 1200 mg day, or 1000 to about 1200 mg a day, or 1200 to about 1600 mg a day. In certain embodiments of the invention, the amount of ribavirin administered to a patient concurrently with pegylated interferon-α can be for example from about 8 to about 15 mg per kilogram per day, typically about 8, 12 or 15 mg per kilogram per day, in divided doses.

Those of skill in the art understand that embodiments of the invention include administering interferon-α and ribavirin either alone or in combination in methods for obtaining patient-specific regimen responsiveness profiles and then using the regimen responsiveness profiles to design optimal therapeutic regimens for patients suffering from pathological conditions such as Hepatitis C infections. In addition, there are a number of other HCV therapeutic agents known in the art in addition to interferon-α and ribavirin that can be administered either alone or in combination with interferon-α and/or ribavirin in order to obtain patient-specific regimen responsiveness profiles and then using the regimen responsiveness profiles to design optimal therapeutic regimens for patients suffering from pathological conditions such as Hepatitis C infections. Such anti-viral agents include for example, but are not limited to, immunomodulatory agents, such as thymosin; VX-950, CYP inhibitors, amantadine, and telbivudine; Medivir's TMC435350, GSK 625433, R1626, ITMN 191, other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (see, e.g., compounds of U.S. Pat. Nos. 5,807,876, 6,498,178, 6,344,465, 6,054,472, WO 97/40028, WO 98/40381, WO 00/56331 the contents of which are incorporated by reference, and mycophenolic acid and derivatives thereof, and including, but not limited to VX-497, VX-148, and/or VX-944); or combinations of any of the above. A variety of such inhibitors which may be used in these methods are known in the art and described below (see, e.g. Sheldon et al., Expert Opin Investig Drugs. 2007 August; 16(8):1171-81).

In some embodiments of the invention, a therapeutic agent used in methods to obtain a patient-specific regimen responsiveness profile (and to optionally use this profile to design an optimized therapeutic regimen) is VX-950. VX-950 (also termed (Telaprevir) is an orally active targeted antiviral therapy for hepatitis C virus (HCV) infection that has been shown to reduce plasma HCV RNA in patients with genotype 1 virus (see, e.g. U.S. Patent Nos. 20070218138 and 20060089385, the contents of which are incorporated by reference). In some embodiments, the dose of amorphous VX-950 can be a standard dose, e.g., about 1 g to about 5 g a day, more typically about 2 g to about 4 g a day, more typically about 2 g to about 3 g a day, e.g., about 2.25 g or about 2.5 g a day. For example, a dose of about 2.25 g/day of amorphous VX-950 can be administered to a patient, e.g., about 750 mg administered three times a day. Such a dose can be administered, e.g., as three 250 mg doses three times a day or as two 375 mg doses three times a day. In some embodiments, the 250 mg dose is in an about 700 mg tablet. In some embodiments, the 375 mg dose is in an about 800 mg tablet. As another example, a dose of about 2.5 g/day of amorphous VX-950 can be administered to a patient, e.g., about 1250 mg administered two times a day. As another example, about 1 g to about 2 g of amorphous VX-950 a day can be administered to a patient, e.g., about 1.35 g of amorphous VX-950 can be administered to a patient, e.g., about 450 mg administered three times a day. Vertex Pharmaceuticals Incorporated has disclosed results from an ongoing Phase 2b study evaluating Telaprevir-based treatment in patients with genotype 1 chronic hepatitis C virus (HCV) infection who did not achieve sustained virologic response (SVR) with at least one prior pegylated interferon (peg-IFN) and ribavirin (RBV) regimen. In this study, 52% (60 of 115; intent-to-treat analysis) of patients randomized to receive treatment with a 24-week Telaprevir-based regimen (12 weeks of Telaprevir in combination with peg-IFN and RBV, followed by 12 weeks of peg-IFN and RBV alone) maintained undetectable HCV RNA 12 weeks post-treatment (SVR12).

In some embodiments of the invention, a therapeutic agent used in methods to obtain a patient-specific regimen responsiveness profile (and to optionally use this profile to design an optimized therapeutic regimen) is SCH 503034. SCH 503034 is another hepatitis C virus (HCV) protease inhibitor (see, e.g. U.S. Patent Nos. 20070224167, 20060281688, 20070185083, 20070099825, and Sarazzin et al., Gastroenterology. 2007 April; 132(4):1270-8. Epub 2007, the contents of which are incorporated by reference). Illustrative dosing regimens for SCH 503034 include 200 mg, 300 mg, or 400 mg, 3 times daily orally. For example, genotype-1 patients in a 14-day course of treatment (5 treatment arms including 1 placebo arm), showed an HCV RNA reduction with the maximum HCV reduction of more than 2 logs in the group receiving 400 mg of SCH503034. SCH503034 was safe and well-tolerated with no serious adverse events. Schering-Plough Corporation disclosed results from an analysis of a Phase II trial of Boceprevir which showed a high rate of sustained virologic response (SVR) in patients receiving Boceprevir-based combination therapy in a study of 595 treatment-naïve patients with chronic hepatitis C virus (HCV) genotype 1. In a 48-week treatment regimen, the SVR rate at 12 weeks after the end of treatment (SVR 12) was 74 percent (ITT) in patients who received 4 weeks of PEGINTRON (peginterferon alfa-2b) and REBETOL® (ribavirin, USP) prior to the addition of Boceprevir (800 mg TID) (P/R lead-in), compared to 38 percent for patients in the control group receiving 48-weeks of PEGIntron And REBETOL alone. Patients in the study who received 48-weeks of Boceprevir in combination with PEGIntron And REBETOL from the beginning of treatment, (no PegIntron/ribavirin (P/R) lead-in) achieved 66 percent SVR 12. In the two 28-week Boceprevir arms of the study, SVR at 24 weeks after the end of treatment (SVR 24) was 56 percent and 55 percent for patients in the lead-in and no lead-in arms, respectively. Importantly, for patients who received the PEGIntron And REBETOL lead in and had rapid virologic response (RVR), defined as undetectable virus (HCV-RNA) in plasma after 4 weeks of Boceprevir treatment, SVR (ITT) was 82 percent in the 28-week regimen and 92 percent in the 48 week regimen. See also, Njoroge et al. Acc Chem. Res. 2008 January; 41(1):50-9.

In some embodiments of the invention, a therapeutic agent used in methods to obtain a patient-specific regimen responsiveness profile (and to optionally use this profile to design an optimized therapeutic regimen) is Medivir's TMC435350 (see, e.g. the disclosure presented at the 14th International Symposium on Hepatitis C Virus and Related Viruses in Glasgow, Scotland by Simmen et al. entitled “Preclinical Characterization of TMC435350, a novel macrocyclic inhibitor of the HCV NS3/4A serine protease”, the contents of which are incorporated by reference). This disclosure demonstrates the ability of TMC435350 to reduce the amount of Hepatitis C virus replication in laboratory replicon experiments via protease inhibition. In addition, this disclosure notes that combinations of TMC435350 with interferon is also reported to enhance RNA reduction (>4 logs reduction in the replicon model), and to suppress the appearance of drug-resistance. Results presented at 43rd annual meeting of the European Association for the Study of the Liver show that TMC435350 was well tolerated during 5 days of dosing, and provoked a strong and rapid antiviral activity in genotype 1 infected individuals. See, e.g. Reesink et al., Safety of the HCV protease inhibitor TMC435350 in healthy volunteers and safety and activity in chronic hepatitis C infected individuals: a phase I study, 43rd annual meeting of the European Association for the Study of the Liver (EASL 2008), Milan, 2008.

In some embodiments of the invention, a therapeutic agent used in methods to obtain a patient-specific regimen responsiveness profile (and to optionally use this profile to design an optimized therapeutic regimen) is ITMN 191 (see, e.g. U.S. Patent Application No. 20050267018, the contents of which are incorporated by reference). InterMune reports that dosing in a Phase 1a single ascending-dose (SAD) trial of ITMN-191 in healthy subjects shows no serious adverse events were reported in the SAD trial. Preliminary safety data from the SAD trial provide evidence that ITMN-191 was well tolerated and safe at the doses intended for the Phase 1b multiple-ascending dose of ITMN-191. InterMune additionally reported that, based on a preliminary review of the available and still blinded clinical data from the four completed cohorts of the Phase 1b study, ITMN-191 was safe and well-tolerated.

In some embodiments of the invention, a therapeutic agent used in methods to obtain a patient-specific regimen responsiveness profile (and to optionally use this profile to design an optimized therapeutic regimen) is GSK 625433. A study presented at the 42nd annual meeting of the European Association for the Study of the Liver (EASL 2007) disclosed GSK625433 as a highly potent and selective inhibitor of genotype 1 HCV polymerases that is observed to be synergistic with interferon-in vitro.

In some embodiments of the invention, a therapeutic agent used in methods to obtain a patient-specific regimen responsiveness profile (and to optionally use this profile to design an optimized therapeutic regimen) is Taribavirin. Taribavirin (formerly known as viramidine) is an oral pro-drug of ribavirin that is less likely to cause anemia. In a study presented at the 43rd annual meeting of the European Association for the Study of the Liver (EASL 2008) in Milan, investigators disclosed results from an open-label Phase IIb trial, 278 treatment-naive patients with genotype 1 chronic hepatitis C stratified by body weight and baseline viral load and randomly assigned (1:1:1:1) to receive taribavirin at doses of 20, 25, or 30 mg/kg/day, or else weight-based ribavirin (800, 1000, 1200, or 1400 mg/day), all administered with pegylated interferon alfa-2b (PegIntron). Baseline patient characteristics were generally similar across the study arms with regard to factors predictive of treatment response.

In some embodiments of the invention, a therapeutic agent used in methods to obtain a patient-specific regimen responsiveness profile (and to optionally use this profile to design an optimized therapeutic regimen) is a nucleoside having anti-HCV properties, such as those disclosed in WO 02/51425 (4 Jul. 2002), assigned to Mitsubishi Pharma Corp.; WO 01/79246, WO 02/32920, WO 02/48165 (20 Jun. 2002), and WO2005/003147 (13 Jan. 2005) (including R1656, (2′R)-2′-deoxy-2′-fluoro-2′-C-methylcytidine, methylcytidine, shown as compounds 3-6 on page 77) assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), US2005/0038240 (Feb. 17, 2005) and WO2006021341 (2 Mar. 2006), including 4′-azido nucleosides such as R1626, 4′-azidocytidine, assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003); US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004); the content of each of which is incorporated herein by reference in its entirety. For example, patients given oral doses of R1626, (500 mg, 1500 mg, 3000 mg, 4500 mg) achieved viral load reductions of 1.2, 2.6, and 3.7 log 10 in the 100 mg, 300 mg and 4500 mg doses respectively. R1626 was generally well-tolerated with increasing adverse events at the highest dose (4500 mg). No viral resistance was found. Investigators disclosed data on R1626 at the 43rd annual meeting of the European Association for the Study of the Liver (EASL) showing that R1626 produces good response with pegylated interferon/ribavirin and has high barrier to resistance. See, e.g. Nelson et al., High End-of-Treatment Response (84%) After 4 Weeks of R1626, Peginterferon Alfa-2a (40 kd) and Ribavirin Followed By a Further 44 Weeks of Peginterferon Alfa-2a and Ribavirin. 43rd annual meeting of the European Association for the Study of the Liver (EASL 2008), Milan 2008; and Pogam et al., Low Level of Resistance, Low Viral Fitness and Absence of Resistance Mutations in Baseline Quasispecies May Contribute to High Barrier to R1626 Resistance in Vivo. 43rd annual meeting of the European Association for the Study of the Liver (EASL 2008), Milan, 2008.

In some embodiments of the invention, a therapeutic agent used in methods to obtain a patient-specific regimen responsiveness profile (and to optionally use this profile to design an optimized therapeutic regimen) is R71278, a polymerase inhibitor developed by Roche and Pharmasset. With R71278, there is a dose-dependent antiviral activity across all dosing arms with the 1,500 mg twice-daily arm achieving a great than 99% decrease in HCV RNA (viral load). R7128 is reported to be generally safe and well-tolerated with no serious adverse events or any dose reductions due to adverse events. Pharmasset, Inc. has disclosed results of a clinical trial evaluating R7128 1000 mg twice daily (BID) in combination with the standard of care (SOC), Pegasys plus ribavirin, in 31 treatment-naive patients chronically infected with hepatitis C virus (HCV) genotype 1. See, e.g. Lalezari et al., Inhibitor R7128 with Peg-IFN and Ribavirin: Interim Results of R7128 500 mg BID for 28 Days. 43rd annual meeting of the European Association for the Study of the Liver (EASL 2008), Milan, 2008.

Methods for formulating the interferon, ribavirin and other therapeutic agent compositions of the invention for pharmaceutical administration are known to those of skill in the art. See, for example, Remington: The Science and Practice of Pharmacy, 19^{th} Edition, Gennaro (ed.) 1995, Mack Publishing Company, Easton, Pa. Typically the therapeutic agents used in the methods of the invention combined with at pharmaceutically acceptable carrier. The term “pharmaceutically acceptable carrier” is used according to its art accepted meaning and is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration.

Therapeutic compositions of cytokines such as interferon-α and compounds such as ribavirin can be prepared by mixing the desired cytokine having the appropriate degree of purity with optional pharmaceutically acceptable carriers, excipients, or stabilizers in the form of lyophilized formulations, aqueous solutions or aqueous suspensions (see, e.g. Remington: The Science and Practice of Pharmacy Lippincott Williams & Wilkins; 21 edition (2005), and Ansel's Pharmaceutical Dosage Forms and Drug Delivery Systems Lippincott Williams & Wilkins; 8th edition (2004)). For example, pharmaceutical compositions of pegylated interferon alpha-suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benzylalcohol), and surfactants (e.g. tween or polysorabates) in sterile water for injection. Acceptable carriers, excipients, or stabilizers are typically nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Solutions or suspensions used for administering a cytokine can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

Suitable carriers for formulations of interferons in liquid form include, but are not limited to, water, saline solution, buffered solutions, blood, glucose, concentrated plasma, concentrated or fractioned blood, glycerol or any combination thereof. Acceptable excipients or stabilizers that may be added to interferon formulations are nontoxic to recipients at the dosages and concentrations employed, and include buffers and preservatives typically used in the art. The formulations herein may also comprise other active molecules as necessary for the particular indication being treated. A person with ordinary skill in the art is capable of selecting active molecules with complementary activities that do not adversely affect each other in amounts that are effective for the purpose intended. In different embodiments, the formulation may also include bioactive agents including, neurotransmitter and receptor modulators, anti-inflammatory agents, anti-viral agents, anti-tumor agents, antioxidants, anti-apoptotic agents, nootropic and growth agents, blood flow modulators and any combinations thereof.

In addition, the interferon may be included in a sustained release composition. The interferons may, for example, be entrapped in a microsphere prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in, for example, Remington's Pharmaceutical Sciences, Lippincott Williams & Wilkins; 21 edition (May 1, 2005). Alternatively, the interferons may be incorporated into semipermeable matrices of biodegradable solid polymers. The matrices may be in the form of shaped articles, e.g., films, rods, or pellets. Suitable materials for sustained-release matrices include, but are not limited to, poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof. Polymers such as ethylene-vinyl acetate and lactic acid-glycolic acid enable release of molecules for over 100 days. Processes for preparing sustained-release compositions are well known and are described, for example, in U.S. Pat. No. 6,479,065.

### Exemplary Algorithms for Determining Patient-Specific Pharmacokinetic and Pharmacodynamic Parameters:

In certain embodiments of the invention, one or more algorithms is used to obtain a regimen responsiveness profile. Typically, an algorithm is used to determine patient-specific parameters such as the in vivo concentrations of therapeutic agent(s) administered to a patient, the baseline viral load, liver fibrosis or cirrhosis, or presence (e.g. in the serum of the patient) of markers associate with a pathological condition such as alanine transaminase (ALT) or aspartate transaminase (AST). The algorithm(s) can further be used to design an optimized therapeutic regimen (e.g. an interferon dose that is, for example, calculated to avoid severe side effects typically associated with interferon therapy). In embodiments of the invention, the patient may then be tested a plurality of times for the interferon serum concentration or the viral load or any other relevant parameters known to those of ordinary skill in the art. A plurality of patient-specific pharmacokinetic and pharmacodynamic parameters may be obtained by fitting the pharmacokinetic and pharmacodynamic models known in the art (and described herein) to this data. In addition, a wide variety of statistical techniques known and used in the art, such as for example, linear or non-linear regressions, may be employed in embodiments of the invention. In some embodiments, the models or their solutions in analytical or numerical form may be combined or substituted into each other as is commonly done by artisans skilled in this technology.

In certain embodiments of the invention, a first therapeutic regimen can include an initial dose of an agent such as interferon-α and/or ribavirin that, while therapeutically effective, is calculated to avoid substantial adverse side effects, and can be determined by one with ordinary skill in the art from experience, population data, journal articles, etc. By way of non-limiting example, regular interferon-α 2a can be administered at a dosing rate of 3 million international units (MIU) three times per week, whereas interferon-α 2b may be administered at a higher dosing rate of 6 MIU/day. In addition, peginterferon α-2a such as Pegasys® is typically administered in a fixed dose of 180 micrograms (mcg) per week while peginterferon-α-2b such as Pegintron® is typically administered weekly in a weight-based dose of 1.5 mcg per kilogram (thus in a range of 75 to 150 mcg per week). By way of a non-limiting example of a therapeutic regimen comprising multiple therapeutic agents, patients can also receive 1000-1600 mg/day oral ribavirin by mouth daily based upon weight (e.g. 1000 mg/day if weight 75 kg; 1200 mg/day if weight >75 kg etc.). Of course, a person with ordinary skill in the art will undoubtedly appreciate that these specific doses for interferon-α and ribavirin are provided only as a benchmark, and such person will be capable of customizing them depending on patient specific factors. Such factors may include, but are not limited to, patient's response to therapy, patient's ability to tolerate high dosage of interferon, viral genotype, viral kinetics, whether the patient was a prior non-responder or a treatment-naïve, extent of virus, and so forth.

In certain embodiments of the invention, it can be advantageous to vary the dose in order to obtain better estimates of the pK and pD parameters as well as to determine whether these parameters have changed. In some embodiments, interferon may be administered by more than one method, i.e., bolus injection and continuous infusion. In other embodiments, different routes of administration may be employed, such as, subcutaneous bolus and intravenous bolus. In yet other embodiments, the amount of interferon may be changed, such as, administering interferon at a different dosing rate or different concentration. The dose may be varied at any time during the therapy, such as hours, days, weeks or even months after commencement of therapy.

The terms “pharmacodynamic models” and “pharmacodynamic parameters” as used herein also include viral kinetic models and viral kinetic parameters. Various models to estimate Hepatitis C viral kinetics have been developed, and may be used for methods described herein. Examples of suitable viral kinetic models include, but are not limited to, models disclosed in the following references: Alan S. Perelson, et al. (2005). “New kinetic models for the hepatitis C virus.” Hepatology 42(4): 749-754. Andrew H Talal, et al. (2006). “Pharmacodynamics of PEG-IFN α Differentiate HIV/HCV Coinfected Sustained Virological Responders from Nonresponders.” Hepatology 43(5): 943-953. Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21. Dixit, N. M., J. E. Layden-Almer, et al. (2004). “Modelling how ribavirin improves interferon response rates in hepatitis C virus infection.” Nature 432(7019): 922. Neumann, A. U., N. P. Lam, et al. (1998). “Hepatitis C viral dynamics in vivo and the antiviral efficacy of interferon-alpha therapy.” Science 282(5386): 103-7. Powers, et al. (2003). “Modeling viral and drug kinetics: hepatitis C virus treatment with pegylated interferon alfa-2b.” Semin Liver Dis 23 Suppl 1: 13-18. Powers, K. A., R. M. Ribeiro, et al. (2006). “Kinetics of hepatitis C virus reinfection after liver transplantation.” Liver Transpl 12(2): 207-16; Bonate, P. L. (2006). Pharmacokinetic-Pharmacodynamic Modeling and Simulation. New York, Springer Science&Business Media; Gabrielsson, J. and D. Weiner (2000); and Pharmacokinetic and Pharmacodynamic Data Analysis: Concepts and Applications. Stockholm, Swedish Pharmaceutical Press.

By way of non-limiting example, one suitable pharmacokinetic model, a standard 1-compartment model, is presented below:

$$\begin{matrix}\frac{d D}{d t} = Q - {k}_{a} D & ( 1 ) \\ \frac{d C}{d t} = ( \frac{{k}_{a}}{{V}_{d}^{′}} ) D - {k}_{e} C & ( 2 )\end{matrix}$$

By way of non-limiting example, one suitable pharmacodynamic model, known as Hill's equation, is presented below:

$$ɛ ( t ) = \frac{{C ( t )}^{n}}{{EC}_{50}^{n} + {C ( t )}^{n}} \qquad ( 3 )$$

wherein:

D represent dose of interferon in the infusion site (IU);

Q represents infusion rate of interferon (IU/hour);

kₐ represent interferon absorption rate constant (1/hour);

kₑ represents interferon elimination rate constant (1/hour);

V_{d}′ represents apparent volume of distribution (mL);

C represents plasma concentration of interferon (IU/mL);

EC₅₀ represents concentration at which drug's efficacy is half its maximum (IU/mL);

n represents Hill's coefficient which determines how steeply the efficacy rises with increasing concentration; and represents actual efficacy.

Aspects of these equations are described in the art, for example in Powers, et al. (2003). “Modeling viral and drug kinetics: hepatitis C virus treatment with pegylated interferon alfa-2b.” Semin Liver Dis 23 Suppl 1: 13-18 and Perelson, et al. (2005). “New kinetic models for the hepatitis C virus.” Hepatology 42(4): 749-754.

By way of non-limiting example, one suitable viral kinetic model is presented below:

$$\begin{matrix}\frac{d T}{d t} = s + rT ( 1 - \frac{T + I}{{T}_{max}} ) - dT - β VT & ( 4 ) \\ \frac{d I}{d t} = β VT + rI ( 1 - \frac{T + I}{{T}_{max}} ) - δ I & ( 5 ) \\ \frac{d V}{d t} = ( 1 - ɛ ) pI - cV & ( 6 )\end{matrix}$$

### Wherein:

T represents the concentration of uninfected target cells (cells/ml);

I represents the concentration of infected target cells (cells/ml);

Tₘₐₓ represents a maximum size of the liver (cells/ml)

V represents viral load (IU/ml);

s represents a constant rate of uninfected target cells production (cell ml⁻¹*day⁻¹);

r represents maximum specific proliferation rate of infected and uninfected target cells (day⁻¹);

β represents the infection rate constant rate (ml*day⁻¹*IU⁻¹);

p represents virion production rate constant (IU*cell⁻¹*day⁻¹);

c represents virion clearance rate constant (day⁻¹);

δ represents the specific death for infected target cells (day⁻¹);

d represents the specific death rate for uninfected target cells (day⁻¹); and

ε represents overall drug efficacy, i.e. actual efficacy.

Aspects of these equations are described in the art, for example in Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21; and Dahari et al., Curr Hepat Rep. 2008; 7(3): 97-105.

In typical embodiments of the invention, efficacy is defined as the ability of a drug to produce a desired therapeutic effect or a clinical outcome. The efficacy of interferon treatment may be described in terms of overall efficacy (ε), in terms of blocking virion production (εₚ) or in terms of reducing new infections (η). Efficacy may also indicate the rate of sustained virological response, early virological response, rapid virological response, and so forth.

The term “actual efficacy” means an efficacy achieved by administering to a patient an interferon dose. The actual efficacy may be calculated from the clinical outcome, such as interferon serum concentration or viral load data. The term “critical efficacy” means a critical value of efficacy such that for efficacies above the critical value the virus is ultimately cleared, while for efficacies below it, a new chronically infected viral steady-state level is reached. The term “desired efficacy” means a value of efficacy that is estimated to result in a desired clinical outcome including, for example, desired value of, rate of change of, or trend of change in viral load, number of infected target cells, number of uninfected target cells and so forth. The desired efficacy is typically set to maximize the difference between the actual efficacy and the critical efficacy while minimizing the side effects on the patient.

Efficacy of interferon may be varied by varying the dosing rate of interferon. The term “dosing rate” as contemplated herein depends on a quantity of interferon delivered over time, and may be optimized by changing interferon's administration rate or interferon's concentration. In addition, the term “dosing rate” as used herein may also depend on a quality of interferon, and may be changed by switching to a more potent interferon formulation. The dosing rate may be varied rapidly or gradually from one constant rate to another, or according to an approximately sinusoidal function.

The actual efficacy may be determined using various models. In some embodiments, it may be determined using equations 1-3 and C(t) data. Alternatively, it may be calculated by fitting the following equation to V(t) data:

V(t)=V_{bar}[1−ε+εe^{-ct}] (7)

### Wherein:

V(t) represents viral load (IU/ml);

V_{bar} represents initial viral load (IU/ml);

ε represents actual efficacy;

t represents time (day); and

c represents clearance constant (day⁻¹).

Aspects of these equations are described in the art, for example in Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21; and Dahari et al., Curr Hepat Rep. 2008; 7(3): 97-105.

By way of non-limiting example, the critical efficacy may be estimated using the following equations:

$${ɛ}_{c} = 1 - \frac{c ( δ {T}_{max} + r {\overline{T}}_{0} - {rT}_{max} )}{p β {T}_{max} {\overline{T}}_{0}} \qquad ( 8 )$$

wherein T̅ₒ is a number of uninfected target cells at uninfected steady state (I=V=0) which may be represented as:

$${\overline{T}}_{0} = \frac{{T}_{max}}{2 r} [ r - d + \sqrt{{( r - d )}^{2} + \frac{4 rs}{{T}_{max}}} ] \qquad ( 9 )$$

wherein r>d and s≦dTₘₐₓ so T̅ₒ≦Tₘₐₓ and the other variables are those disclosed in the above equations (e.g. p represents virion production rate constant (IU*cell⁻¹*day⁻¹)).

Aspects of these equations are described in the art, for example in Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21; and Dahari et al., Curr Hepat Rep. 2008; 7(3): 97-105.

Because the values of at least some parameters in equation (9) may be difficult to determine, especially in real time, the value of critical efficacy may not always be determined in real time. Instead, the limits of critical efficacy (ε_{crit}) may be presented as a function of time as follows: if t=0, then 0<ε_{crit}<1 but if t>0, then L(t)<ε_{crit}<1 wherein L(t) is a lower limit for critical efficacy that fit the real-time V(t) data. In order to estimate L(t), the viral kinetic model parameters may be determined in real time by fitting the viral kinetic model to the viral load data.

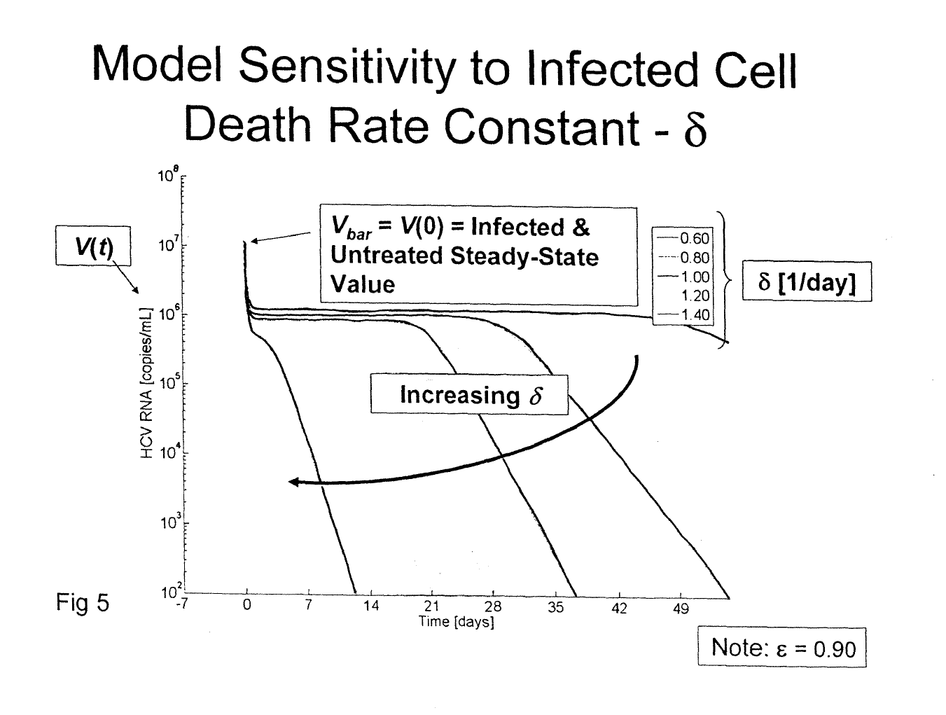

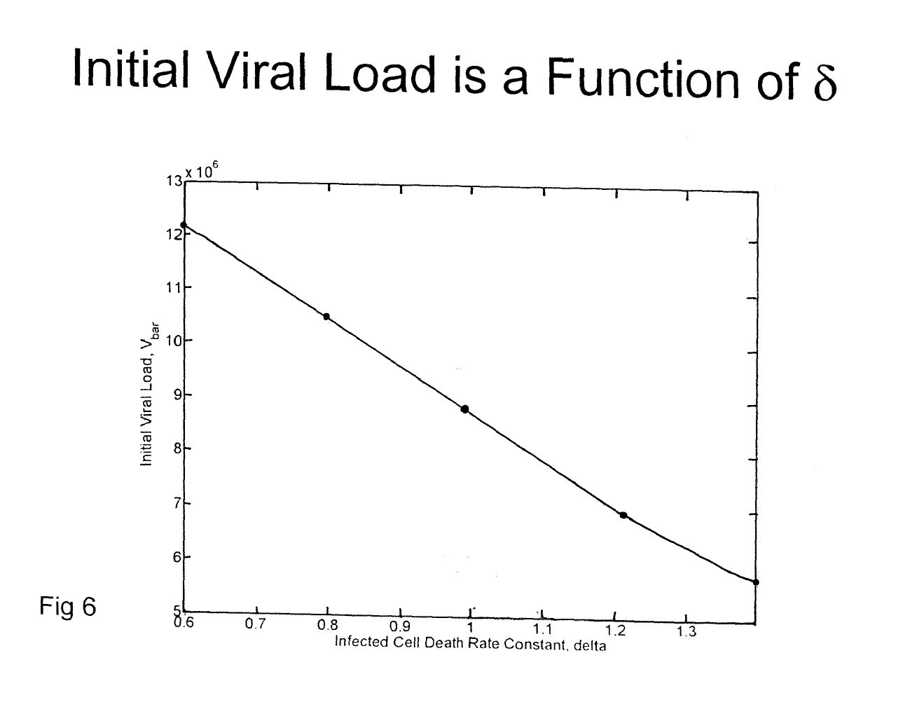

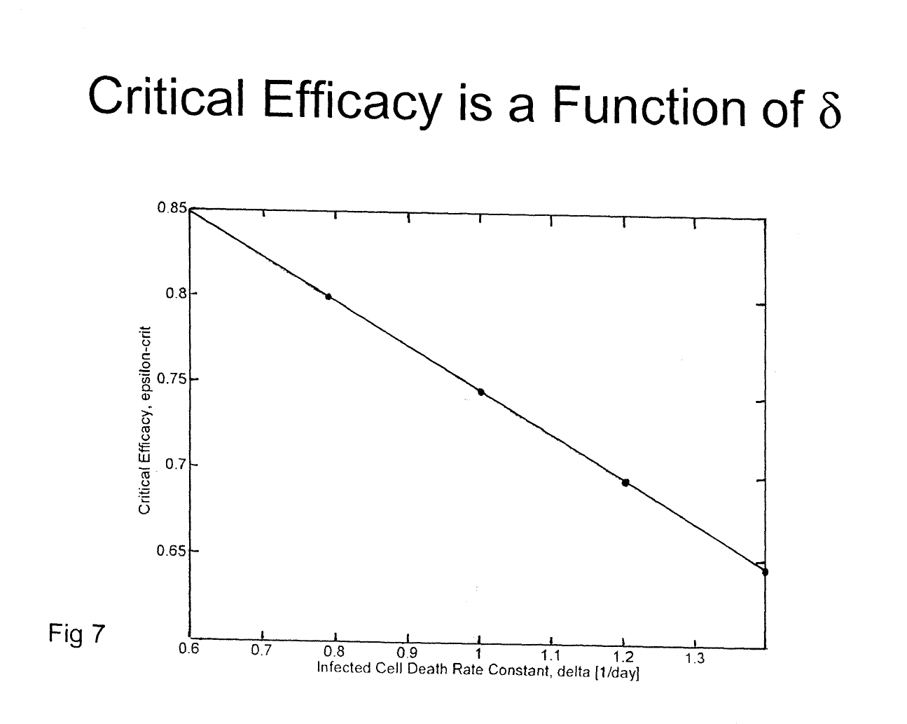

To better illustrate this point, for the following example only, it will be assumed that all parameters in equation 5, except the specific death rate for infected target cells (δ) and critical efficacy are known. As shown in FIG. 5, patients with a low value of δ who begin therapy with a majority of their target cells being infected may exhibit a plateau in their viral load before they eventually clear the virus. As shown in FIGS. 6 and 7, δ is typically inversely proportional to initial viral load and critical efficacy.

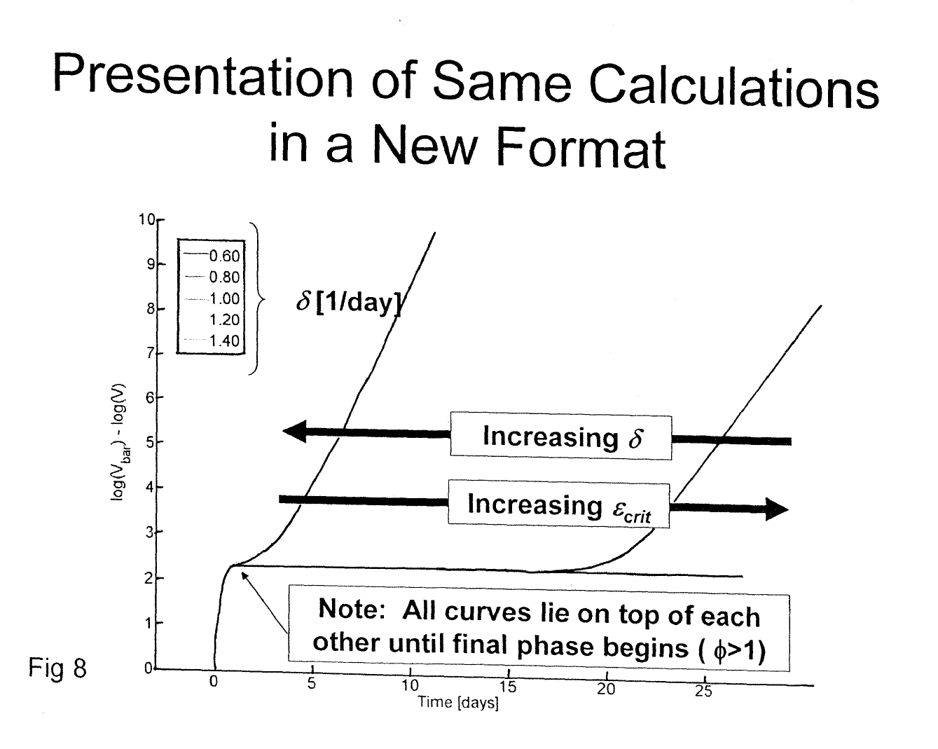

Accordingly, as shown in FIG. 8, δ can not be determined until the final phase begins (φ>1) because all curves lie on top of each other. Thus critical efficacy cannot be estimated until the final phase. If the decline in viral load reaches a plateau, L(t) can be calculated as a function of time from fitting a model for Hepatitis C viral kinetics. Referring to FIG. 8, by day 5, patients with δ equal to or greater than 1.4 would have entered the final phase, whereas patients with δ of 0.6, 0.8, 1.0, and 1.2 are still in the flat stage. Accordingly, if on day 5, a patient's viral tests exhibit that he or she is still in the flat stage, this patient's δ is less than 1.4. Then, L(5) for this patient may be calculated by substituting 1.4 for δ into equation 5. Similarly, L(5) may be calculated by substituting 1.2 for δ into equation 5.

Once L(t) is calculated, it may be used to determine the desired efficacy and to determine whether the actual efficacy needs to be adjusted. If the actual efficacy is less than L(t), then it may be desirable to increase it to ensure that the actual efficacy is equal or greater than ε_{crit}. If, however, the actual efficacy is greater than L(t), it may be kept constant or decreased. It should be understood that even if the actual efficacy is greater than L(t), it may be further increased, if feasible, to maximize the difference between the actual efficacy and L(t), as described above. Viral load response to the change in the dosing rate, and thus actual efficacy, may be monitored and that information may be used to determine new efficacy and L(t). The steps described above may be repeated to continue increasing or decreasing actual efficacy as necessary or as tolerated.

To achieve the desired efficacy, the controller may calculate necessary changes in the dosing rate using the pK and pD models based on information received from concentration feedback loop, viral load feedback loop, or both. In embodiments using only concentration feedback loop, the efficacy may be represented as a function of concentration, which is a function of the dosing rate Q(t). In embodiments using viral load feedback loop, Q(t) and efficacy may be represented as a function of pharmacodynamic parameters such as a viral load, number of infected target cells number of uninfected target cells, and so forth. Once the changes in the dosing rate are implemented, the controller may use the models and the data from the feedback loops to optimize the dosing rate so the actual efficacy equals the desired efficacy.

Those of skill in this art understand that although some pK or pD parameters may be determined in a matter of hours or days, determining other parameters may require data taken over longer periods of time such as weeks or months. In addition, many of the pK and pD parameters as well as the structure and complexity of the model may change during the therapy. Accordingly, the blood samples for determination of pK and pD parameters may be taken throughout the therapy. More specifically, the samples may be taken from 0 to 48 weeks after commencement of therapy. Typically, the blood samples may be taken more frequently around the peak and less frequently around the tail. Furthermore, the duration of sampling may also depend on the type of interferon used as well as on the individual's response to therapy. In one specific embodiment, the samples for determination of may be taken at 0, 2, 4, 6, 8, 10, 12, 16, 20, 24, 36, 38, 40, 42, 44, 46, 48, 52, 56, 60, 72, 96, 120, 144, and 168 hours during week 1, and then at week 2, 4, 8, 16, 24, 36 and 48. In another embodiment, samples are taken every week up to week 48 or 72. Data for concentration and viral load may be obtained according to the same or different schedule. It will also be understood that samples may be taken more frequently in order to provide adequate feedback to the controller, and these samples may also be used to determine or optimize the pK and pD parameters.

Embodiments of the invention comprise administering interferon at a dosing rate that results in high efficacy during an initial stage (e.g. a first or second therapeutic regimen) followed by a dosing rate that results in decreased efficacy for the follow-up stage (e.g. a second or third therapeutic regimen). Applicants discovered that increasing efficacy of interferon over time instead of decreasing it may result in a number of unexpected benefits. These unexpected benefits include, but are not limited to, decrease in duration of therapy, limiting duration of patient exposure to higher doses of interferon, limiting the side effects, and decreasing the cost of therapy.

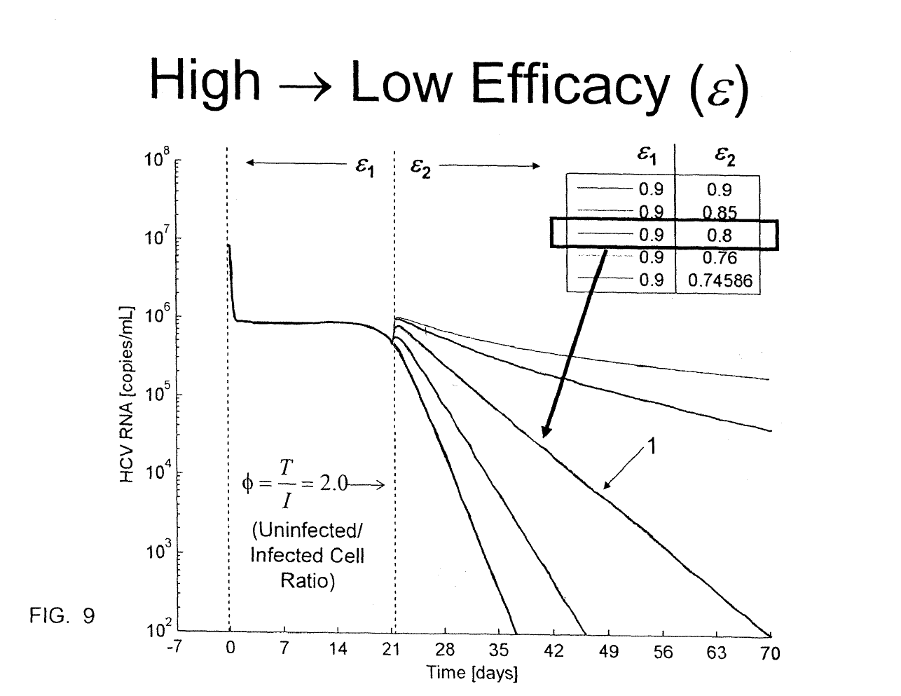

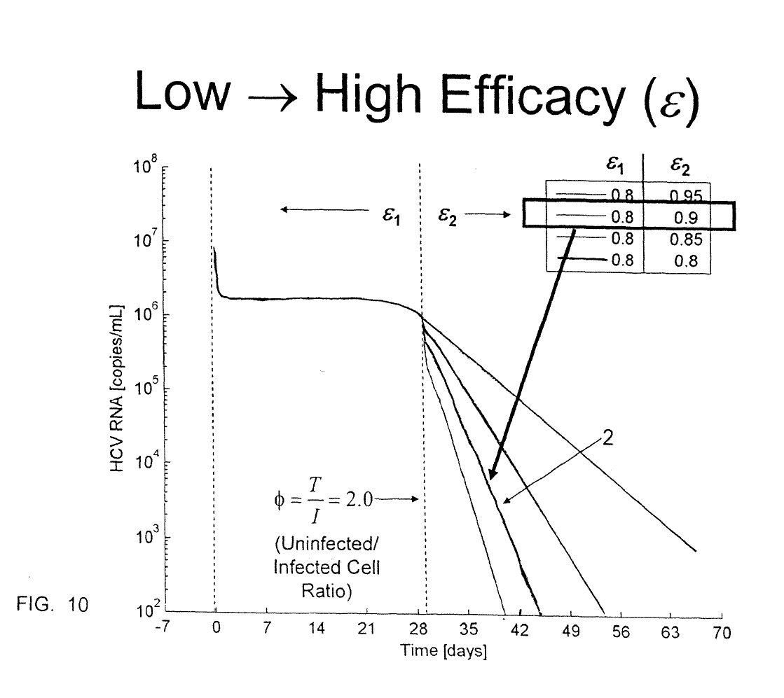

Some potential benefits of the Applicants' methods may be demonstrated in reference to FIGS. 9 and 10, which model therapeutic regimens according to embodiments disclosed herein, respectively. The graphs in FIG. 9 and FIG. 10 were generated using a viral kinetic model, which is presented in equations disclosed herein, and using an exemplary set of model parameters. One with ordinary skill in the art would undoubtedly realize that these graphs predict a response to treatment of hypothetical patients, and are presented here simply as an illustrative example. The following parameters were used: d=0.01 day⁻¹, p=2.9 IU/cell/day, β=2.25*10⁷ ml*day⁻¹*IU⁻¹, δ=1.0 day⁻¹, c=6.0 day⁻¹, r=2.0 day⁻¹, s=1.0 cell*ml⁻¹*day⁻¹, and Tₘₐₓ=3.6*10⁷ cells*ml⁻¹. To generate the graph in FIG. 9, the actual interferon efficacy was set to 0.9 during the first stage of the treatment and lowered to 0.8 during the second stage. To generate the graph in FIG. 10, the actual efficacy was set to 0.8 during the first stage and increased to 0.9 during the second stage. For example, curve 1 in FIG. 9 shows that the patient is exposed to the higher dose for 21 days while the total duration of treatment is 70 days. On the contrary, when the values of the actual efficacy are reversed, as depicted in curve 2 in FIG. 10, the patient is exposed to the higher dose for only 16 days while the total duration of treatment is 45 days, and thus the patient also receives a lower total overall dose of interferon. By way of non-limiting example, the dosing rate during the first stage, i.e. dosing rate that results in lower efficacy, may be set to about 3 to 9 MIU/day (based on a 75 kg patient), and the dosing rate during the second stage may be set to about 9 MIU/day to 20 MIU/day.

One of ordinary skill in the art can appreciate that in various embodiments of the invention, the dosing rates may be dependent or independent of each other. If dependent, the dosing of the first stage may be set to fall between about 5 to 95%, or about 20% and 80%, or about 20 and 50%, or about 25% of the dosing rate of the second stage (dosing rate resulting in a higher efficacy). The second stage may last for the remainder of the therapy or, alternatively, may be followed by one or more additional stages. The efficacy during the additional stages may be higher or lower than the efficacy during the second stage. However, in the typical embodiment, the second stage of the therapy would always provide a higher level of the actual efficacy as compared to the actual efficacy during the first stage of the therapy.

### Exemplary Computer System Embodiments of the Invention

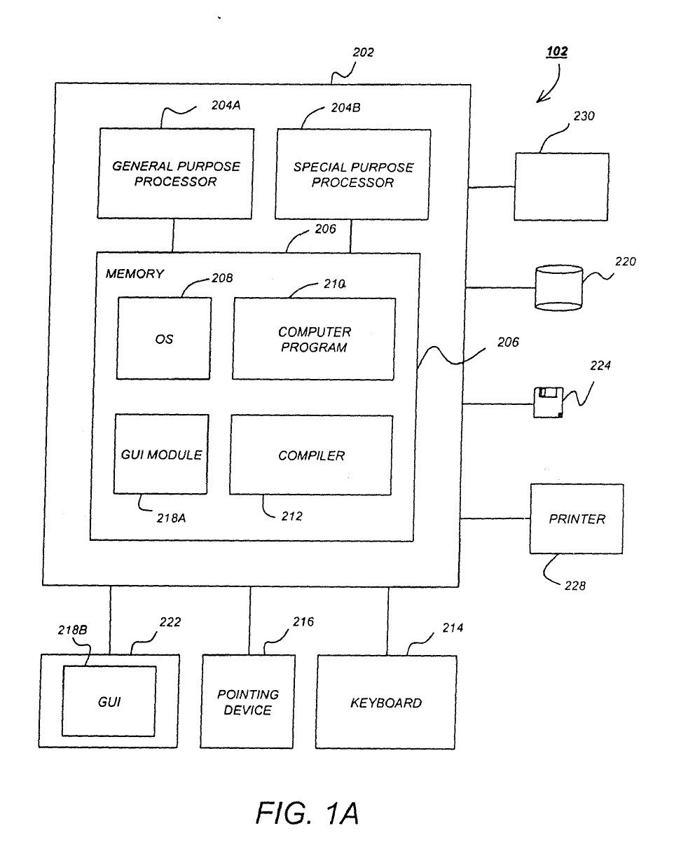

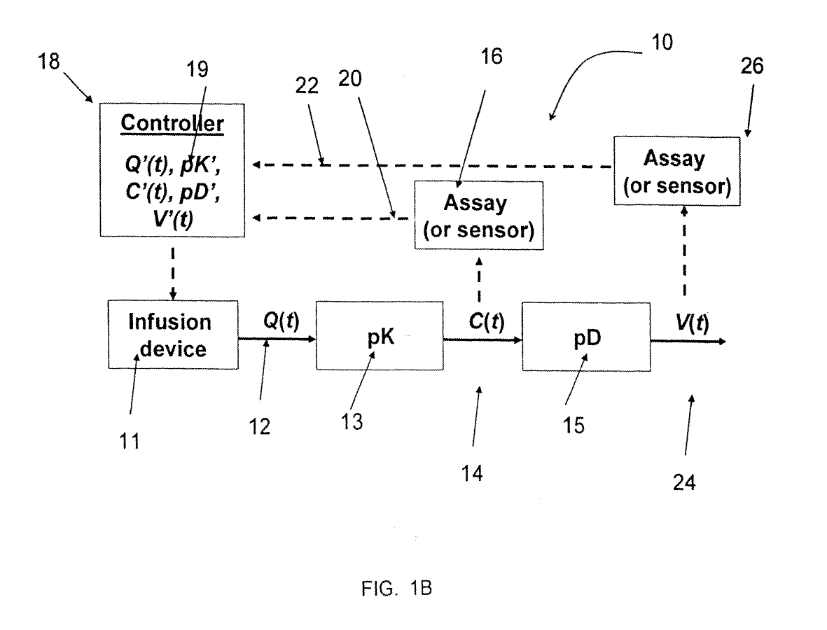

Embodiments of the invention disclosed herein can be performed for example, using one of the many computer systems known in the art (e.g. those associated with medication infusion pumps). FIG. 1A illustrates an exemplary generalized computer system 202 that can be used to implement elements the present invention, including the user computer 102, servers 112, 122, and 142 and the databases 114, 124, and 144. The computer 202 typically comprises a general purpose hardware processor 204A and/or a special purpose hardware processor 204B (hereinafter alternatively collectively referred to as processor 204) and a memory 206, such as random access memory (RAM). The computer 202 may be coupled to other devices, including input/output (I/O) devices such as a keyboard 214, a mouse device 216 and a printer 228.

In one embodiment, the computer 202 operates by the general purpose processor 204A performing instructions defined by the computer program 210 under control of an operating system 208. The computer program 210 and/or the operating system 208 may be stored in the memory 206 and may interface with the user 132 and/or other devices to accept input and commands and, based on such input and commands and the instructions defined by the computer program 210 and operating system 208 to provide output and results. Output/results may be presented on the display 222 or provided to another device for presentation or further processing or action. In one embodiment, the display 222 comprises a liquid crystal display (LCD) having a plurality of separately addressable liquid crystals. Each liquid crystal of the display 222 changes to an opaque or translucent state to form a part of the image on the display in response to the data or information generated by the processor 204 from the application of the instructions of the computer program 210 and/or operating system 208 to the input and commands. The image may be provided through a graphical user interface (GUI) module 218A. Although the GUI module 218A is depicted as a separate module, the instructions performing the GUI functions can be resident or distributed in the operating system 208, the computer program 210, or implemented with special purpose memory and processors.

Some or all of the operations performed by the computer 202 according to the computer program 110 instructions may be implemented in a special purpose processor 204B. In this embodiment, the some or all of the computer program 210 instructions may be implemented via firmware instructions stored in a read only memory (ROM), a programmable read only memory (PROM) or flash memory in within the special purpose processor 204B or in memory 206. The special purpose processor 204B may also be hardwired through circuit design to perform some or all of the operations to implement the present invention. Further, the special purpose processor 204B may be a hybrid processor, which includes dedicated circuitry for performing a subset of functions, and other circuits for performing more general functions such as responding to computer program instructions. In one embodiment, the special purpose processor is an application specific integrated circuit (ASIC).

The computer 202 may also implement a compiler 212 which allows an application program 210 written in a programming language such as COBOL, C++, FORTRAN, or other language to be translated into processor 204 readable code. After completion, the application or computer program 210 accesses and manipulates data accepted from I/O devices and stored in the memory 206 of the computer 202 using the relationships and logic that was generated using the compiler 212. The computer 202 also optionally comprises an external communication device such as a modem, satellite link, Ethernet card, or other device for accepting input from and providing output to other computers.

In one embodiment, instructions implementing the operating system 208, the computer program 210, and the compiler 212 are tangibly embodied in a computer-readable medium, e.g., data storage device 220, which could include one or more fixed or removable data storage devices, such as a zip drive, floppy disc drive 224, hard drive, CD-ROM drive, tape drive, etc. Further, the operating system 208 and the computer program 210 are comprised of computer program instructions which, when accessed, read and executed by the computer 202, causes the computer 202 to perform the steps necessary to implement and/or use the present invention or to load the program of instructions into a memory, thus creating a special purpose data structure causing the computer to operate as a specially programmed computer executing the method steps described herein. Computer program 210 and/or operating instructions may also be tangibly embodied in memory 206 and/or data communications devices 230, thereby making a computer program product or article of manufacture according to the invention. As such, the terms “article of manufacture,” “program storage device” and “computer program product” as used herein are intended to encompass a computer program accessible from any computer readable device or media.

Of course, those skilled in the art will recognize that any combination of the above components, or any number of different components, peripherals, and other devices, may be used with the computer 202. Although the term “user computer” is referred to herein, it is understood that a user computer 102 may include portable devices such as medication infusion pumps, analyte sensing apparatuses, cellphones, notebook computers, pocket computers, or any other device with suitable processing, communication, and input/output capability.

FIG. 1B presents a specific illustrative embodiment system 10 for performing methods disclosed herein. The interferon may be administered at a dosing rate Q(t) 12 from an infusion device 11 including, but not limited to, a pump, a depot, an infusion bag, or a syringe. Once the therapy is commenced, the interferon serum concentration 14, represented as C(t), may be determined by sampling a patient's blood by assay or sensor 16, and communicated to a controller 18, as represented by a concentration feedback loop 20. In addition to or instead of loop 20, the system 10 may also include a viral load feedback loop 22. According to the loop 22, patient's viral load 24, represented as V(t), may be determined by sampling patient's blood by assay or sensor 26 and may be communicated to the controller 18. Based on C(t), V(t) or both, controller 18 may calculate the dosing rate 12, which may then be adjusted if necessary either automatically by the controller or manually by an individual administering the therapy. In addition, patient-specific pK parameters 13 and pD parameters 15 may be determined from this data. Although the controller 18 may be a conventional process controller such as a PID controller, one can also utilize an adaptive model predictive process controller or model reference adaptive control. In general, a model predictive controller may be programmed with mathematical models of a “process” to predict “process” response to proposed changes in the inputs. These predictions are then used to calculate appropriate control actions. In response to control actions, the model predictions are continuously updated with measured information from the “process” to provide a feedback mechanism for the controller. In addition, the mathematical models may be continuously optimized to match the performance of the “process.”

In the system shown in FIG. 1B, the controller 18 may be programmed with patient-specific pK or pD parameters, population or subpopulation averages, or a combination thereof together with pharmacokinetic and pharmacodynamic models to calculate the dosing rate necessary to achieve desired clinical outcome. During the therapy, the controller continuously processes the data received from the feedback loops to optimize the dosing rate based on a patient's response to the therapy. In some embodiments, the controller 18 may also manipulate the pharmacokinetic and pharmacodynamic parameters, as well as the mathematical models based on concentration and viral load data to adopt or customize the models for individual patients and specific conditions. Patient-specific pharmacokinetic parameters may be determined by fitting pharmacokinetic models to concentration data whereas Patient-specific pharmacodynamic parameters may be determined by fitting the kinetic model, such as described by equations 1-3, to viral load data V(t).

In FIG. 1B, the controller 18 may use patient-specific pharmacokinetic or pharmacodynamic parameters, population or subpopulation averages, or combination thereof together with pharmacokinetic, pharmacodynamic, or viral kinetic models to calculate the dosing rate for desired efficacy based on C(t), V(t) or both. In FIG. 1B, pK refers to the physical pharmacokinetic system of a real patient. On the other hand, the parameter pK 19 refers to the pharmacokinetic model and parameter values used by the controller to describe pK, and which may be drawn from the real patient, population, or subpopulation averages. Similar notation is used for pD, C, V and Q.

In an embodiment of a system 10 having the loop 22 only, a given patient is assumed to have a set of individual pharmacokinetic parameters, represented as pK, and thus actual efficacy may be represented as a function of concentration, which is a function of the dosing rate Q(t). The controller 18 may use pharmacokinetic and pharmacodynamic models to calculate the suitable dosing rate for desired efficacy based on the concentration or other physiological characteristic data. Such models are known and are disclosed in, for example, Bonate, P. L. (2006). Pharmacokinetic-Pharmacodynamic Modeling and Simulation. New York, Springer Science&Business Media; Andrew H Talal, et al. (2006). “Pharmacodynamics of PEG-IFN α Differentiate HIV/HCV Coinfected Sustained Virological Responders from Nonresponders.” Hepatology 43(5): 943-953′ Gabrielsson, J. and D. Weiner (2000). Pharmacokinetic and Pharmacodynamic Data Analysis: Concepts and Applications. Stockholm, Swedish Pharmaceutical Press.

### Exemplary Applications for System Embodiments of the Invention:

As noted above, embodiments of the invention can be used to determine patient-specific pharmacokinetic and pharmacodynamic parameters and then construct patient-specific interferon delivery profiles. These patient-specific delivery profiles can then be used to design patient-specific therapeutic regimens. As briefly discussed below, a variety of factors can be considered and adapted to these embodiments of the invention.

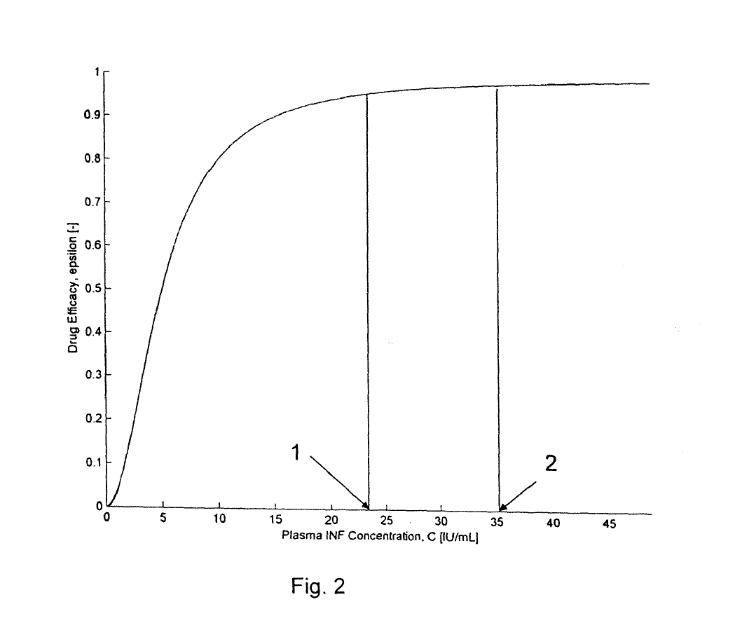

In one embodiment, Q(t) may be controlled to maintain desired interferon serum concentration. For example, although interferon efficacy is dependent on interferon serum concentration, this dependency is not linear, but is represented by a sigmoid function. Accordingly, as shown in FIG. 2, increasing interferon concentration from point 1 to point 2 does not result in a significant increase in efficacy. Such increase may, however, result in more severe adverse effects and thus, result in increased rate of patient noncompliance. Thus, in this example, the set-point for concentration may be set to the lowest concentration resulting in a desired efficacy, determined as described in detail below. The controller then may set Q(t) to a value calculated using equations 1 and 2 to maintain the desired concentration. During the therapy, the concentration feedback loop enables the controller to continually adjust Q(t) to maintain interferon concentration at the set-point.

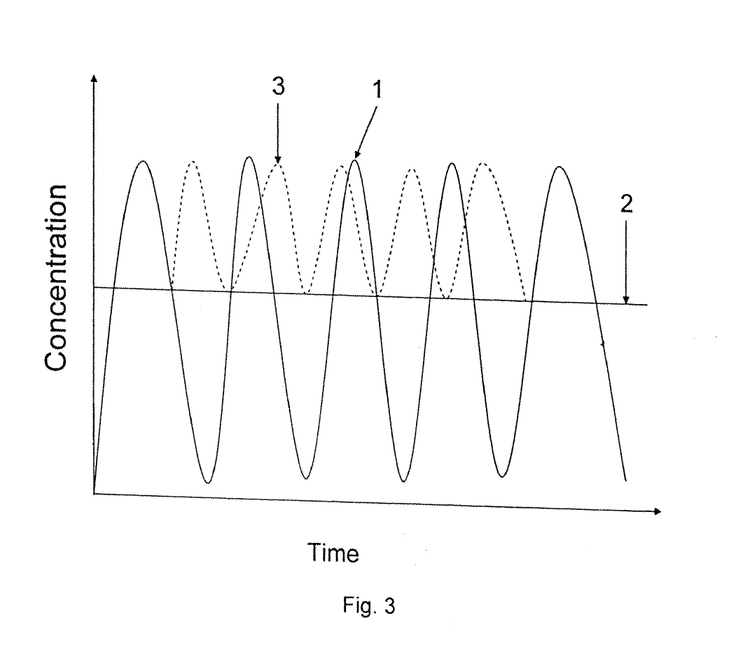

In embodiments where a therapeutic agent such as interferon is administered in an intermittent manner, optimal times and amount for additional infusions may be determined. Typically, interferon serum concentration reaches its maximum shortly after interferon is injected, and then declines over time, as shown by curve 1 in FIG. 3. To maintain interferon serum level, and thus efficacy at a desired level, additional interferon may be administered when the interferon serum concentration falls below a certain value, concentration set-point, represented by line 2 in FIG. 3. Accordingly, the controller may calculate the time for and the amount of the additional interferon infusion so the concentration does not fall below the set-point. In one embodiment, the interferon may be administered by a bolus injection shortly before the concentration falls to the set-point, as represented by curve 3 in FIG. 3.

By way of non-limiting example, the concentration set-point may be greater than EC₅₀ (the drug concentration at which drug's effectiveness is half its maximum), determined from pD models. Typically, the concentration set-point may be chosen so the difference between the concentration and EC₅₀ is maximized without exposing the patient to severe adverse side effects. In other embodiments, the concentration set-point is chosen so the actual efficacy is higher than the critical efficacy, as explained in detail below. Again, typically, this difference is maximized while avoiding severe side effects. A person with ordinary skill in the art would undoubtedly be able to balance the need for maximizing likelihood of positive clinical outcome with the need to not expose the patient to adverse side effects, and thus ensure patient compliance with therapy. Cost of the therapy may also be considered by one with ordinary skill in the art when selecting the concentration set-point.

Typically, HCV RNA levels exhibit a biphasic or triphasic decline in response to therapy. In a biphasic response, viral load rapidly declines during the first phase, and gradually declines during the second phase. In a triphasic response, a rapid initial decline in the viral load is followed by “shoulder phase”—in which viral load decays slowly or remains constant—and a third phase of resumed viral decay. See Dahari, H., A. Lo, et al. (2007). “Modeling hepatitis C virus dynamics: liver regeneration and critical drug efficacy.” J Theor Biol 247(2): 371-81. (hereinafter the “Dahari, 2007 reference”). In addition, some patients may exhibit a more complex pattern such as for example, a rebound in viral load after the first stage, or a change in the rate of decline in the middle of the second phase. Throughout this application, the term “phase” is used to refer to changes in viral load kinetics. On the other hand, the term “stage” is used to refer to changes in the dosing rate or efficacy. The phases and stages may or may not correspond to one another. In certain embodiments of the invention, one can maintain interferon (or other agent) efficacy at different levels, e.g. administer interferon at different dosing rates, at different phases of HCV RNA decline. The controller may use pharmacokinetic and pharmacodynamic parameters and models to predict a point in time when different phases are expected to occur, and change the dosing rate accordingly.

Efficacy of an interferon may be varied by varying the dosing rate of interferon, which is described in detail below. The term “dosing rate” as contemplated herein depends on a quantity of interferon delivered over time, and may be optimized by changing interferon's administration rate or interferon's concentration. In addition, the term “dosing rate” as used herein may also depend on a potency of interferon, and may be changed by switching to a more potent interferon formulation. The dosing rate may be varied rapidly or gradually from one constant rate to another, or according to an approximately sinusoidal function. In some embodiments, it may be advantageous to increase the dosing rate gradually, i.e., according to an approximated ramp function, in order to minimize adverse side effects by allowing the patient to acclimate to a higher dosing rate. Alternatively, especially if a patient is tolerant to interferon, it may be desirable to increase the dosing rate rapidly, i.e. according to an approximated step function, in order to maximize time at a higher dosing rate.

In one embodiment, interferon may be administered at a high dosing rate during the first stage, and at a low dosing rate during the second stage. Alternatively, the dosing rate may be increased over time. In addition, the dosing may be changed more than once over the course of the therapy. In one embodiment of the invention, the method comprises administering interferon at a first dosing rate, and a second dosing rate, wherein the second dosing rate is higher than the first dosing rate, and thus results in a higher efficacy than the first dosing rate. The method may further comprise administering interferon at one or more follow-up dosing rates which may result in a lower efficacy than the second dosing rate. The method may also employ follow-up dosing rates that may be calculated based on patient's response to the therapy up that point.

The initial dosing rate is preferably administered for between about 1 and 120 days. Alternatively, the second dosing rate commences when the ratio of uninfected to infected target cells is of the order of magnitude of 1, equal to 1, or greater than 1. The dosing rate of interferon may be optimized by changing the rate of interferon administration, the concentration of interferon, or employing a more potent interferon formulation. In another embodiment of the invention, the method comprises administering interferon at an initial dosing rate, and then adjusting the dosing rate based on the patient's response to the initial interferon administration. That is, in such embodiments of the invention, the inventors have contemplated an individualized interferon therapeutic regimen for treating Hepatitis C virus infection based on a patient's pharmacokinetic and pharmacodynamic parameters. Generally, the actual efficacy, i.e. the efficacy calculated from the clinical outcome following the administration of a known interferon dose, and the lower limit of a critical efficacy, i.e. a critical value of efficacy such that for efficacies above the critical value the virus is ultimately cleared, while for efficacies below it, a new chronically infected viral steady-state level is reached. Subsequently, the actual efficacy may be adjusted to values higher than the lower limit of critical efficacy. In at least this embodiment of the invention, the difference between the actual efficacy and the lower limit of critical efficacy is maximized.

In some embodiments of the invention, the treatment regimen comprises two stages. For example, a dosing rate can be increased once from a first dosing rate to a second dosing rate, and then be maintained at the second dosing rate for the remainder of the therapeutic regimen. Alternatively, the treatment regimen may comprise more than two stages. In such embodiments, after the initial increase from the first dosing rate to the second dosing rate, the dosing rate may be changed again, i.e., the second stage may be followed by at least one follow-up stage. The follow-up dosing rates, and thus actual efficacy at each follow-up stage, may be higher or lower than the dosing rates, and actual efficacy, at the preceding stages.

In another embodiment of the invention, a method of treating hepatitis virus infection is provided where the method generally involves adjusting the dosing rate of interferon, and thus the actual efficacy, based on a patient's response to administration of interferon at initial or previous dosing rate. Increasing actual efficacy over the value for the critical efficacy may decrease the duration of all phases of viral load decline: the initial phase, the plateau phase, and the final phase; therefore the duration of the entire therapy would decrease. In addition, Applicants believe that the optimal clinical results may be achieved when the actual efficacy is higher than the critical efficacy throughout the therapy in order for the patients to effectively clear the virus and/or maintain a non-pathological measurements of viral load. In at least one such embodiment of the invention, the difference between actual efficacy and critical efficacy is preferably maximized.

The patient-specific treatment regimens described herein provide for optionally measuring such patients' parameters as the baseline viral load or other parameters associated with Hepatitis C virus, which are described in more detail below. The regimen then provides for administration of interferon at a dosing rate preferably calculated to avoid severe side effects typically associated with interferon therapy. The patient may then be tested for the interferon serum concentration or the viral load or any other relevant parameters known to those of ordinary skill in the art to infer the actual efficacy. Based on the results of these tests and respective comparison of the baseline values, actual efficacy and critical efficacy may be estimated. Critical efficacy may be estimated from a patient's response to the initial dosing rate using various viral kinetics models. Then, the initial interferon dosing rate is adjusted to a second dosing rate where the actual efficacy is greater than or equal to the estimated critical efficacy. This process can be repeated as necessary for the duration of the therapy.

In at least one embodiment, before initiating a first therapeutic regimen, a patient may be tested for their baseline viral load or any other parameter associated with Hepatitis, such as, for example, liver fibrosis or cirrhosis, or presence in serum of markers such as alanine transaminase (ALT) or aspartate transaminase (AST), among others. An interferon therapy may then be initiated at a therapeutically effective dosing rate. Preferably, the initial dosing rate of interferon while therapeutically effective is calculated to avoid substantial adverse side effects, and can be determined by one with ordinary skill in the art from experience, population data, journal articles, etc.

The duration of stages of a therapeutic regimen may be defined in terms of time or in terms of decline in the viral load. In some embodiments, the therapeutic regimen may be concluded when a patient's viral load stays at 10² International Units per Milliliter (IU/ml) or less, or 10² RNA copies/ml or less for about 4 weeks, or at lowest detection limit of the assay for 4 weeks. By way of non-limiting example, in embodiments where the interferon is administered in a first therapeutic regimen, the first stage may last for about 1 to 120 days, typically between about 21 and 35 days, and optionally about 28 days. In various embodiments, the second stage may last between about 0 and 30 days, for example between about 14 and 30 days. In other embodiments, the second stage may be followed by at least one more stage with an increased or decreased efficacy for the total treatment time of 24 weeks or 48 weeks. Alternatively, the initial stage may last until a 1-log or a 2-log reduction in viral load is measured. After the initial stage, the dosing rate may be increased and kept constant for the remainder of the therapy, or may be adjusted at least once again.

By way of non-limiting example, in embodiments where the interferon is administered at a high dosing rate, the first stage may last for about 3 to 5 weeks, and typically for about 4 weeks. In other embodiments, the first stage may last until HCV RNA level is between about the lower detection limit of the employed assay and 10⁷ IU/ml, 10 IU/ml and 10⁷ IU/ml, about 100 IU/ml and 10⁷ IU/ml, or about 10³ UI/ml and 10⁷ IU/ml. Typically, the detection limit of the assay is about 10 to 100 IU/ml. In yet other embodiments, the first stage may last until a 2-log reduction, a 3-log reduction, or a 4-log reduction in the viral load is achieved. The second stage may last for about 42 to 52 weeks, typically for about 48 weeks. Alternatively, the second stage may last until HCV RNA is equal to or less than about 10² IU/ml, 10 copies/ml, or stays below the detection limit of the employed assay for about 4 weeks. The dosing rate may also be reduced multiple times, such as, for example, at 2 log reduction, then at 3 log reduction, and then at a 4 log reduction in HCV RNA levels for the remainder of the therapy.

In yet other embodiments, the duration of stages may be defined in terms of ratio of infected target cells to uninfected target cells. In one embodiment, the duration of stages may be defined in terms of ratio of infected target cells to uninfected target cells. It has been shown that not all hepatocytes (liver cells) may be intrinsically susceptible to hepatitis virus infection. On the contrary, cells other than hepatocytes, i.e. cells other than the ones that reside in the liver, may be susceptible to hepatitis virus infection. See Powers, K. A., R. M. Ribeiro, et al. (2006). “Kinetics of hepatitis C virus reinfection after liver transplantation.” Liver Transpl 12(2): 207-16. Accordingly, the term “target cells” means cells that are susceptible to hepatitis virus infection regardless of whether they are hepatocytes or other cell types.

Studies have shown that patients who begin therapy with a majority of the target cells infected are predicted to exhibit a plateau or shoulder phase in their viral load before they eventually clear the virus. It was hypothesized that the plateau phase is attributed to the time required for the uninfected target cells to outnumber the infected target cells. The ratio of number of uninfected target cells (T) to the number of infected target cells (I) is defined as φ (phi). Accordingly, for patients with advanced hepatitis who begin therapy with majority of their target cells infected, φ is less than 1, almost 0. Once the first stage of the therapy is initiated, φ begins to increase in value and would ideally move toward infinity towards the end of the therapy. In this context, in one embodiment of the invention, φ, T, or I may be determined by fitting models of viral kinetics for Hepatitis C, as is described herein.

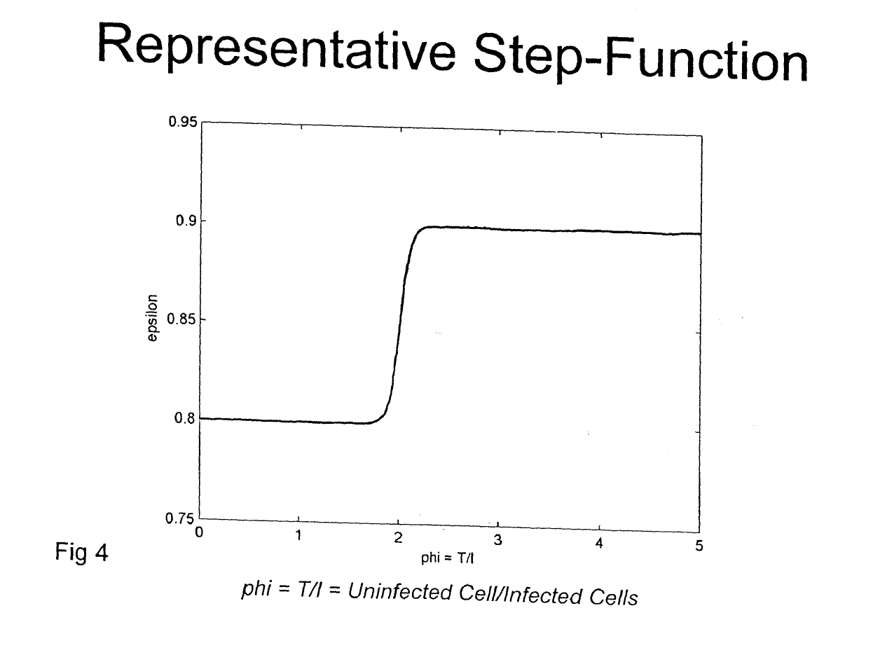

In some embodiments of the invention, the dosing rate of interferon may be increased when φ is on the order of magnitude of greater than, or equal to 1, or when φ is equal to 2, as shown in FIG. 4. Kinetic models provide evidence that φ is greater than 1 when the patients viral load enters its final decline. Accordingly, in one embodiment viral load may be monitored to determine when the final decline phase begins, and the dosing rate may be increased once the patient appears to enter the final decline phase.

Patient-specific pK and pD parameters may also be used to predict ultimate viral response to therapy. Because interferon therapy is expensive and may result in adverse side effects, it may be desirable to predict lack of sustained viral response in the early stages of therapy. For example, it has been shown that interferon efficacy during the early stages of therapy may be indicative of the ultimate success of the therapy. See Layden, et al. (2002). “First Phase Viral Kinetic Parameters as Predictors of Treatment Response and Their Influence on the Second Phase Viral Decline.” J Viral Hep 9, 340-345. In addition, it has been shown that EC, is likely to be lower in patients who ultimately exhibit a sustained response, than in patients who exhibit no response. Furthermore, the median therapeutic quotient, i.e. ratio of average drug concentration and EC₅₀ (C_{ace}/EC₅₀), and minimum interferon plasma concentration are likely to be higher in patients who ultimately exhibit a sustained response, than in patients who exhibit no response. See Andrew H Talal, et al. (2006). “Pharmacodynamics of PEG-IFN α Differentiate HIV/HCV Coinfected Sustained Virological Responders from Nonresponders.” Hepatology 43(5): 943-953.

Thus, in order to predict whether a specific therapy will result in a sustained viral response, the patient-specific pK and pD parameters, which may be determined as described above, may be compared to population or sub-population averages for similar conditions and therapies. If the patient's parameters indicate that the sustained response is not likely, the therapy regimen is typically adjusted in the early stages. In one specific embodiment, the therapy may be adjusted if the actual efficacy of interferon is less than 98%. In another specific embodiment, in a PEG-IFN α-2b therapy, the therapy may need to be adjusted if a patient's EC50 value is equal to or greater than 0.04 μg/L. Additionally, the PEG-IFN α-2b therapy may need to be adjusted if the median therapeutic quotient is less than 10.1 μg/L after the first week of treatment or is less than 14 μg/L after the second week of treatment. In yet other embodiments of a PEG-IFN α-2b therapy, the therapy may need to be adjusted if the PEG-IFN α-2b interferon concentration is less than 2.8 μg/L after the first week of treatment or 5.4 μg/L after the second week of treatment.

In another embodiment, effectiveness of different therapeutic agents may be compared to select the optimal drug for a specific patient. First, different therapeutic agents can be administered to a patient in order to determine patient-specific pharmacokinetic parameters for each agent. Second, Q(t) may be adjusted so the C(t) profiles of each of the therapeutic agents are equivalent, and efficacy of each drug may be determined as described above. Third, the therapeutic agents with superior efficacy profile may be used for the therapy. Alternatively, if several therapeutic agents have similar efficacy profile, the least expensive agent or the agent causing less side effects may be selected for use in further therapeutic regimens.

### Systems for the Administration of Agents Such as Interferons:

In the therapeutic regimens described herein, therapeutic agents (e.g. interferon) can be administered according to art accepted methodologies. In one exemplary embodiment, interferon may be administered by injection either using a traditional needle and a syringe system, or using a needle-free injection technology. Needle free injection system generally works by forcing liquid medication at high speed through a tiny orifice that is held against the skin. The diameter of the orifice is typically smaller than the diameter of a human hair. This creates an ultra-fine stream of high-pressure fluid that penetrates the skin without using a needle. Examples of needle free systems are disclosed, for example, in U.S. Pat. Nos. 7,320,677 and 7,238,167. In addition, interferon may be administered by a drip from an infusion container.

In another embodiment, the interferon may be delivered from a depot. A “depot” includes, but is not limited to capsules, microspheres, particles, gels, coating, matrices, wafers, pills or other pharmaceutical delivery compositions. An example of suitable non-limiting design of a depot implant is discussed in details in a pending application entitled Drug Depot Implant Designs And Methods Of Implantation, Ser. No. 11/403,733, filed on Apr. 13, 2006.

In one embodiment, interferon is administered in a substantially continuous manner. The term “substantially continuous manner” as contemplated herein means that the dosing rate is constantly greater than zero during the periods of administration. The term includes embodiments when the drug is administered at a steady rate or variable rate, i.e., continuous infusion, as well as when the drug is administered in a series of rapid injections of a fixed amount of the drug in the shortest feasible time interval, such as administering interferon from a pump with a stepper motor. Alternatively, interferon may be administered in an intermittent manner, such as, for example, by bolus injections on an hourly, daily, or weekly basis. In some embodiments, interferon may be administered only in a substantially continuous manner or only in an intermittent manner throughout the entire treatment period. In other embodiments, these manners of interferon administration may be combined during the same stage or altered during different stages of the treatment.

In certain embodiments of the invention, the therapeutic agent is administered from a pump (e.g. so as to be administered in a “substantially continuous manner”). Suitable types of pumps include, but are not limited to, osmotic pumps, interbody pumps, infusion pumps, implantable pumps, peristaltic pumps, other pharmaceutical pumps, or a system administered by insertion of a catheter at or near an intended delivery site, the catheter being operably connected to a pharmaceutical delivery pump. It is understood that pumps can be internal or external as appropriate. It may be advantageous to employ a programmable pump for the methods described herein.

When selecting a suitable pump, a number of characteristics need to be considered. These characteristics include, but are not limited to, biocompatibility (both the drug/device and device/environment interfaces), reliability, durability, environmental stability, accuracy, delivery scalability, flow delivery (continuous vs. pulse flow), portability, reusability, back pressure range and power consumption. While biocompatibility is always an important consideration, other considerations vary in importance depending on the device application. A person with ordinary skill in the art is capable of selecting an appropriate pump for the methods described herein.

A variety of external or implantable pumps may be used to administer the interferon. One example of an external pump is Medtronic MiniMed® pump and one example of a suitable implantable pump is Medtronic SynchroMed® pump, both manufactured by Medtronic, Minneapolis, Minn. In these pumps, the therapeutic agent is pumped from the pump chamber and into a drug delivery device, which directs the therapeutic agent to the target site. The rate of delivery of the therapeutic agent from the pump is typically controlled by a processor according to instructions received from the programmer. This allows the pump to be used to deliver similar or different amounts of the therapeutic agent continuously, at specific times, or at set intervals between deliveries, thereby controlling the release rates to correspond with the desired targeted release rates. Typically, the pump is programmed to deliver a continuous dose of interferon to prevent, or at least to minimize, fluctuations in interferon serum level concentrations.

The interferon may be delivered subcutaneously, intramuscularly, parenterally, intraperitoneally, transdermally, or systemically. In specific embodiments, interferon may be delivered subcutaneously or for a systemic infusion. A drug delivery device may be connected to the pump and tunneled under the skin to the intended delivery site in the body. Suitable drug delivery devices include, but are not limited to, those devices disclosed in U.S. Pat. Nos. 6,551,290 and 7,153,292.

A wide variety of continuous infusion devices known in the art can be used to deliver one or more antiviral agents to a patient infected with HCV. Continuous interferon-α administration may for example be accomplished using an infusion pump for the subcutaneous or intravenous injection at appropriate intervals, e.g. at least hourly, for an appropriate period of time in an amount which will facilitate or promote a desired therapeutic effect. Typically the continuous infusion device used in the methods of the invention has the highly desirably characteristics that are found for example in pumps produced and sold by the Medtronic corporation. In illustrative embodiments of the invention, the cytokine is administered via an infusion pump such as a Medtronic MiniMed model 508 infusion pump. The Model 508 is currently a leading choice in insulin pump therapy, and has a long history of safety, reliability and convenience. Typically the pump includes a small, hand-held remote programmer, which enables diabetes patients to program cytokine delivery without accessing the pump itself.

Alternatively, continuous administration can by accomplished by, for example, another device known in the art such as a pulsatile electronic syringe driver (Provider Model PA 3000, Pancretec Inc., San Diego Calif.), a portable syringe pump such as the Graseby model MS 1 6A (Graseby Medical Ltd., Watford, Herts England), or a constant infusion pump such as the Disetronic Model Panomat C-S Osmotic pumps, such as that available from Alza, may also be used. Since use of continuous subcutaneous injections allows the patient to be ambulatory, it is typical over use of continuous intravenous injections.

Infusion pumps and monitors for use in embodiments of the invention can be designed to be compact (e.g. less than 15×15 centimeters) as well as water resistant, and may thus be adapted to be carried by the user, for example, by means of a belt clip. As a result, important medication can be delivered to the user with precision and in an automated manner, without significant restriction on the user's mobility or life-style. The compact and portable nature of the pump and/or monitor affords a high degree of versatility in using the device. As a result, the ideal arrangement of the pump can vary widely, depending upon the user's size, activities, physical handicaps and/or personal preferences. In a specific embodiment, the pump includes an interface that facilitates the portability of the pump (e.g. by facilitating coupling to an ambulatory user). Typical interfaces include a clip, a strap, a clamp or a tape.

A wide variety of formulations tailored for use with continuous infusion pumps are known in the art. For example, formulations which simulate a constant optimized dose injection, such as, but not limited to, short-acting unconjugated forms of interferon-α as well as long-acting interferon-α-polymer conjugates and various-sustained release formulations, are contemplated for use. Typical routes of administration include parenteral, e.g., intravenous, intradermal, intramuscular and subcutaneous administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. Regimens of administration may vary. Such regimens can vary depending on the severity of the disease and the desired outcome.

Following administration of a interferon-α, and/or ribavirin or other therapeutic agents to a person infected with HCV, the HCV burden in the individual can be monitored in various ways well known to the skilled practitioner familiar with the hallmarks of HCV infection. In the case of chronic hepatitis infection, a therapeutically effective amount of the drug may reduce the numbers of viral particles detectable in the individual and/or relieve to some extent one or more of the signs or symptoms associated with the disorder. For example, as disclosed in detail above, in order to follow the course of hepatitis replication in subjects in response to drug treatment, hepatitis RNA may be measured in serum samples by, for example, an rt-PCR procedure such as one in which a nested polymerase chain reaction assay uses two sets of primers derived from a hepatitis genome. Farci et al., 1991, New Eng. J. Med. 325:98-104. Ulrich et al., 1990, J. Clin. Invest., 86:1609-1614. Histological examination of liver biopsy samples may then be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity.

In another embodiment of the invention, an article of manufacture (e.g. a kit) containing materials useful for the treatment of HCV infection as described above is provided. The article of manufacture can comprise a container and a label. Suitable containers include, for example, continuous infusion pumps, infusion tubing sets, catheters, bottles, vials, syringes, and test tubes. The containers may be formed from a variety of materials such as glass or plastic. The container can hold a composition (e.g. cytokine or other therapeutic composition) which is effective for treating the condition (e.g. chronic hepatitis infection) and may have a sterile access port (for example the container may be an intravenous solution bag or a vial having a stopper pierceable by a hypodermic injection needle). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The pharmaceutical compositions useful in the methods of the invention can be included in a container, pack, or dispenser together with instructions for administration. That result can be reduction and/or alleviation of the signs, symptoms, or causes of a disease or any other desired alteration of a biological system. For example, in a further embodiment of the invention, there are provided kits containing materials useful for treating pathological conditions with interferon. The article of manufacture comprises a container with a label. Suitable containers include, for example, bottles, vials, and test tubes. The containers may be formed from a variety of materials such as glass or plastic. The container holds a composition having an active agent which is effective for treating pathological conditions such as HCV infection. The active agent in the composition is typically interferon-α and/or ribavirin. The label on the container indicates that the composition is used for treating pathological conditions with interferon-α and/or ribavirin.

Those of skill in the art will understand that there are a variety of permutations of the disclosed methods. One could for example, alter the dose or the duration of treatment depending upon aspects of HCV infection such an amount of virions eliminated and/or levels of multi-drug resistance observed in the patient. Certain of the embodiments may be adapted to the treatment of HIV infection, other “interferon-responsive diseases” such as HepB and D, as well as cancers such as leukemia, melanoma, lymphomas, Karposi's sarcoma, MS, chronic granulomatous disease, pulmonary fibrosis, and tuberculosis. In essence, embodiments of the invention can be adapted to any infection where host factors can influence the outcome of one or more therapeutic regimens.

Throughout this application, various journal articles, patents, patent applications, and other publications etc. are referenced (e.g. U.S. Patent No. (see, e.g. U.S. Pat. Nos. 6,172,046; 6,461,605; 6,387,365; and 6,524,570; U.S. Patent Application Nos.: 20060257365; 20070202078; 20050112093; 20050031586; 20030004119; and 20030055013 and Dahari, H., A. Lo, et al. (2007). “Modeling hepatitis C virus dynamics: liver regeneration and critical drug efficacy.” J Theor Biol 247(2): 371-81. Dahari, H., R. M. Ribeiro, et al. (2007). “Triphasic decline of hepatitis C virus RNA during antiviral therapy.” Hepatology 46(1): 16-21; and Dahari et al., Curr Hepat Rep. 2008; 7(3): 97-105.). The disclosures of such publications etc. are hereby incorporated by reference herein in their entireties. The present invention is not to be limited in scope by the embodiments disclosed herein, which are intended as single illustrations of individual aspects of the invention, and any that are functionally equivalent are within the scope of the invention. Further, even though the invention herein has been described with reference to particular examples and embodiments, it is to be understood that these examples and embodiments are merely illustrative of the principles and applications of the present invention. It is therefore to be understood that numerous modifications may be made to the illustrative embodiments and that other arrangements may be devised without departing from the spirit and scope of the present invention as defined by the following claims. Publications describing aspects of this technology include for example, U.S. Pat. Appln. Nos. 2005/0063949 and 2007/0077225; U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101 S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. However, the invention is only limited by the scope of the appended claims. All numbers recited in the specification and associated claims that refer to values that can be numerically characterized with a value other than a whole number (e.g. the concentration of a compound in a solution) are understood to be modified by the term “about”.

### EXAMPLES

### Example 1

### Modeling Interferon Therapies of Various Dosing Regimens

The pharmacokinetics of continuously administered IFN were modeled using a single-compartment model with a depot at the subcutaneous injection site. Complete bioavailability and first-order rates of absorption and elimination were assumed. The pharmacodynamic model published by Dahari, Ribeiro and Perelson (Hepatology 2007; 46:16-21.) was used to model viral kinetics, while the PK and viral kinetic models were coupled using a Hill Equation. Model parameter values were obtained from a literature survey. The resulting set of PK/PD model equations were numerically integrated using a MATLAB computer program, which allowed the rate of continuous IFN delivery to be varied among various specified temporal phases of therapy. The results of the model calculations are summarized in Table I.

TABLE I
Summary Results from the PK/PD Modeling of Various Staged Dosing
Regimens of IFN for Treating HCV.

| Continuous Dosing | | Time to Viral |
|---|---|---|
| Regimen | Dosing Regimen Details | Response* (days) |
| Low Constant Dose | 1.3 MIU/day, indefinitely | 52 |
| High Constant Dose | 10 MIU/day | 28 |
| Induction, Typical | 10 MIU/day for 28 days, 1.3 | 45 |
| | MIU/day indefinitely | |
| Delayed-Induction | 1.3 MIU/day for 28 days, 10 | 32 |
| | MIU/day for 28 days, 1.3 | |
| | MIU/day indefinitely | |
| Delayed-Induction, | 1.3 MIU/day, 10 MIU/day | 32 |
| Shortened Duration | for 14 days, 1.3 MIU/day | |
| | indefinitely | |

*Time to viral response means time required for the viral load to drop below the assumed detection limit of the assay (100 IU/mL), as explained above.

The model calculations provide insight as to why typical induction therapies have not been successful and provide evidence that a “delayed-induction” therapy could potentially be more effective, while minimizing the length of time patients have to endure the side-effects associated with high-dose IFN.

### Example 2

### Modeling of Continuous Interferon Therapy

Modeling parameters described in Example 1 were used to compare repeated bolus injections with continuous infusion of fully biopotent non-pegylated interferon alpha into subcutaneous tissue.

The pharmacokinetic model calculations (plasma IFN concentration vs. time) suggest that continuous IFN administration could result in IFN levels that are more stable than standard dosing regimens of either non-pegylated or pegylated interferon alpha-2b. The relatively constant levels of continuous IFN could potentially mitigate the severity and frequency of flu-like symptoms associated with bolus administration. The pharmacodynamic model calculations (viral load vs. time) suggest that the relatively stable PK profile could help patients avoid viral rebound, potentially improving clinical outcomes. These results provide evidence that continuously administered non-pegylated IFN could potentially become the new backbone of hepatitis C combination therapy.

1. A method of using a patient-specific regimen responsiveness profile obtained from a patient infected with hepatitis C virus (HCV) to make a patient-specific therapeutic regimen, the method comprising:
administering at least one therapeutic agent to the patient following a first therapeutic regimen;
obtaining pharmacokinetic or pharmacodynamic parameters from the patient so as to observe a patient-specific response to the first therapeutic regimen, wherein the pharmacokinetic or pharmacodynamic parameters comprise at least one of:
a concentration of the therapeutic agent in the blood of the patient that results from the first therapeutic regimen; or
a concentration of hepatitis C virus present in the patient;
using the pharmacokinetic or pharmacodynamic parameters observed in the patient in response to the first therapeutic regimen to obtain a patient-specific regimen responsiveness profile; and
using the patient-specific regimen responsiveness profile to make a first patient-specific therapeutic regimen.

2. The method of claim 1, wherein the first therapeutic regimen comprises interferon-α and the first patient-specific therapeutic regimen is selected to:
maintain serum interferon-α concentrations in the patient at a value greater than a EC₅₀, a concentration at which the effectiveness of interferon-α is 50% of its maximum;
maintain serum interferon-α concentrations in the patient at a value where the actual efficacy of interferon-α in the patient is greater than the critical efficacy of interferon-α;
modulate interferon-α concentrations in the patient so that the patient is administered different interferon dosing regimens during different phases of hepatitis C viral load decline;
modulate interferon-α concentrations in the patient so that a difference between the actual efficacy of interferon-α and the critical efficacy of interferon-α in the patient is increased; or
modulate interferon-α concentrations in the patient so as to reduce dose-dependent side effects observed during the administration of interferon-α.

3. The method of claim 1, wherein the first therapeutic regimen comprises interferon-α and the pharmacokinetic or pharmacodynamic parameters for a concentration of administered interferon-α in the blood of the patient are obtained from the patient using an algorithm comprising:
$$\frac{d D}{d t} = Q - {k}_{a} D$$
$$\frac{d C}{d t} = ( \frac{{k}_{a}}{{V}_{d}^{′}} ) D - {k}_{e} C$$
$$or$$
$$ɛ ( t ) = \frac{{C ( t )}^{n}}{{EC}_{50}^{n} + {C ( t )}^{n}}$$
wherein:
D represent dose of interferon in the infusion site (IU);
Q represents infusion rate of interferon (IU/hour);
kₐ represent interferon absorption rate constant (1/hour);
kₑ represents interferon elimination rate constant (1/hour);
Vd′ represents apparent volume of distribution (mL);
C represents plasma concentration of interferon (IU/mL);
EC₅₀ represents concentration at which drug's efficacy is half its maximum value at infinite concentration (IU/mL);
n represents Hill's coefficient; and
ε represents actual efficacy.

4. The method of claim 1, wherein pharmacokinetic or pharmacodynamic parameters for a concentration of hepatitis C virus in the plasma of the patient are obtained from the patient using an algorithm comprising:
$$\begin{matrix}\frac{d T}{d t} = s + rT ( 1 - \frac{T + I}{{T}_{max}} ) - dT - β VT \\ \frac{d I}{d t} = β VT + rI ( 1 - \frac{T + I}{{T}_{max}} ) - δ I \\ \frac{d V}{d t} = ( 1 - ɛ ) pI - cV\end{matrix} \qquad [ EAG 1 ]$$
wherein:
T represents the concentration of uninfected target cells (cells/ml);
I represents the concentration of infected target cells (cells/ml);
Tₘₐₓ represents a maximum size of the liver (cells/ml)
V represents viral load (IU/ml);
s represents a constant rate of uninfected target cells production (cell ml⁻¹*day⁻¹);
r represents maximum specific proliferation rate of infected and uninfected target cells (day⁻¹);
β represents the infection rate constant rate (ml*day⁻¹*IU⁻¹);
p represents virion production rate constant (IU*cell⁻¹*day⁻¹);
c represents virion clearance rate constant (day⁻¹);
δ represents the specific death for infected target cells (day⁻¹);
d represents the specific death rate for uninfected target cells (day⁻¹); and
ε represents overall drug efficacy.

5. The method of claim 1, wherein the actual efficacy of the first therapeutic regimen is determined in the patient is calculated using an algorithm comprising:
V(t)=V_{bar}[1−ε+εe^{-ct}]
Wherein:
V(t) represents viral load (IU/ml);
V_{bar} represents initial viral load (IU/ml);
ε represents actual efficacy;
t represents time (day); and
c represents clearance constant (day⁻¹).

6. The method of claim 1, wherein the first patient-specific therapeutic regimen is initiated when ratio of the number of HCV uninfected target cells to the number of HCV infected cells is greater than or equal to 1.

7. The method of claim 5, wherein the critical efficacy of the first therapeutic regimen is determined in the patient is calculated using an algorithm comprising:
$${ɛ}_{c} = 1 - \frac{c ( δ {T}_{max} + r {\overline{T}}_{0} - {rT}_{max} )}{p β {T}_{max} {\overline{T}}_{0}}$$
wherein T̅ₒ is a number of uninfected target cells at uninfected steady state (I=V=0) which may be represented as:
$${\overline{T}}_{0} = \frac{{T}_{max}}{2 r} [ r - d + \sqrt{{( r - d )}^{2} + \frac{4 rs}{{T}_{max}}} ]$$
wherein r>d and s≦dTₘₐₓ so T̅ₒ≦T_{max.}

8. The method of claim 1, further comprising using the patient-specific profile to assess the patient's likely virological response to a defined interferon-α composition or a defined interferon-α dosing regimen.

9. The method of claim 1, including the further step of observing at least one patient-specific factor comprising:
a level of alanine transaminase, neopterin, 2′,5′ oligo-adenylate synthetase, or aspartate transaminase in plasma of the patient;
a genotype or quasispecies of the hepatitis C virus;
a patient's prior medical treatment history; or
a presence or degree of a side effect that results from the first therapeutic regimen.

10. The method of claim 1, further comprising obtaining pharmacokinetic or pharmacodynamic parameters from the patient so as to observe a patient-specific response to the first patient-specific therapeutic regimen, wherein the pharmacokinetic or pharmacodynamic parameters comprise at least one of:
a concentration of administered interferon-α in the plasma of the patient; or
a concentration of hepatitis C virus in the plasma of the patient;
using the pharmacokinetic or pharmacodynamic parameters observed in the patient in response to the first patient-specific therapeutic regimen to obtain a second patient-specific regimen responsiveness profile; and
using the second patient-specific regimen responsiveness profile to make a second patient-specific therapeutic regimen.

11. The method of claim 1, wherein the first patient-specific therapeutic regimen comprises at least one of: an interferon-α; ribavirin; VX-950; SCH 503034; R1626; or R71278.

12. The method of claim 1, wherein the first patient-specific therapeutic regimen comprises administering interferon-α using a continuous infusion pump.

13. The method of claim 1, wherein the first patient-specific therapeutic regimen comprises administering a first dose of interferon-α during a first phase of hepatitis C viral decline and a second dose of interferon-α during a second phase of hepatitis C viral decline.

14. The method of claim 1, wherein the first patient-specific therapeutic regimen comprises administering a first dose of ribavirin during a first phase of hepatitis C viral decline and a second dose of ribavirin during a second phase of hepatitis C viral decline.

15. The method of claim 1, wherein the first patient-specific therapeutic regimen comprises administering a dose of interferon-α for a period of time selected to maintain a plasma interferon-α concentration above a set-point for the period of time.

16. A method of administering interferon-α to a patient suffering from a Hepatitis C infection, the method comprising:
administering interferon-α to the patient following a first therapeutic regimen;
obtaining pharmacokinetic or pharmacodynamic parameters from the patient to observe a patient-specific response to the first therapeutic regimen wherein the pharmacokinetic or pharmacodynamic parameters comprise at least one of:
a concentration of interferon-α in the blood of the patient that results from the first therapeutic regimen; or
a concentration of hepatitis C virus present in the patient;
using the pharmacokinetic or pharmacodynamic parameters observed in the patient in response to the first therapeutic regimen to make a patient-specific therapeutic regimen;
programming a controller operably coupled to a continuous infusion pump with patient-specific therapeutic regimen information; and
using the continuous infusion pump to administer interferon-α to the patient according to the controller programming.

17. The method of claim 16, wherein the controller is programmed so that the continuous infusion pump administers interferon-α in a manner that:
maintains serum interferon-α concentrations in the patient at a value greater than a EC₅₀, a concentration at which the effectiveness of interferon-α is 50% of its maximum;
maintains serum interferon-α concentrations in the patient at a value where the actual efficacy of interferon-α in the patient is greater than the critical efficacy of interferon-α;
modulates interferon-α concentrations in the patient so that the patient is administered different interferon dosing regimens during different phases of hepatitis C viral load decline;
modulates interferon-α concentrations in the patient so that a difference between the actual efficacy of interferon-α and the critical efficacy of interferon-α in the patient is increased; or
modulates interferon-α concentrations in the patient so as to reduce adverse side effects observed during the administration of interferon-α.

18. The method of claim 16, wherein pharmacokinetic or pharmacodynamic parameters are:
based upon observations of concentrations of interferon-α in the blood of the patient following the first therapeutic regimen; and
are obtained using an algorithm comprising:
$$\frac{d D}{d t} = Q - {k}_{a} D$$
$$\frac{d C}{d t} = ( \frac{{k}_{a}}{{V}_{d}^{′}} ) D - {k}_{e} C$$
$$or$$
$$ɛ ( t ) = \frac{{C ( t )}^{n}}{{EC}_{50}^{n} + {C ( t )}^{n}}$$
wherein:
D represent dose of interferon in the infusion site (IU);
Q represents infusion rate of interferon (IU/hour);
kₐ represent interferon absorption rate constant (1/hour);
kₑ represents interferon elimination rate constant (1/hour);
Vd′ represents apparent volume of distribution (mL);
C represents plasma concentration of interferon (IU/mL);
EC₅₀ represents concentration at which drug's efficacy is half its maximum (IU/mL);
n represents Hill's coefficient; and
ε represents actual efficacy.

19. The method of claim 16, wherein:
the controller is programmed so that the continuous infusion pump administers interferon-α at a dose and for a period of time selected to maintain a plasma interferon-α concentration above a set-point for the period of time; and
the patient-specific therapeutic regimen further comprises administering a nucleoside analog that interferes with Hepatitis C viral replication.

20. A system for administering interferon to a patient having a hepatitis C infection, the system comprising:
a continuous infusion pump having a medication reservoir comprising interferon-α;
a processor operably connected to the continuous infusion pump and comprising a set of instructions that causes the continuous infusion pump to administer the interferon-α to the patient according to a patient-specific therapeutic regimen made by:
administering interferon-α to the patient following a first therapeutic regimen;
obtaining pharmacokinetic or pharmacodynamic parameters from the patient so as to observe a patient-specific response to the first therapeutic regimen wherein the pharmacokinetic or pharmacodynamic parameters comprise at least one of:
a concentration of interferon-α in the blood of the patient that results from the first therapeutic regimen; or
a concentration of hepatitis C virus present in the patient;
using the pharmacokinetic or pharmacodynamic parameters observed in the patient in response to the first therapeutic regimen to obtain a patient-specific regimen responsiveness profile; and
using the patient-specific regimen responsiveness profile to make the patient-specific therapeutic regimen.

21. The system of claim 20, wherein the patient-specific therapeutic regimen maintains plasma interferon-α levels in the patient above 20, 30, 40, 50, 60, 70, 80, 90, 100, 110 or 120 IU/mL.

22. The system of claim 20, wherein the patient-specific therapeutic regimen maintains plasma interferon-α levels in the patient below 140, 130, 120, 110, 100, 90, 80, 70 or 60 IU/mL.

23. The system of claim 20, wherein the interferon-α is not conjugated to a polyol.

24. The system of claim 20, wherein the continuous infusion pump:
has dimensions smaller than 15×15 centimeters; or
is operably coupled to an interface that facilitates the patient's movements while using the continuous infusion pump, wherein the interface comprises a clip, a strap, a clamp or a tape.

25. A program code storage device, comprising:
a computer-readable medium;
a computer-readable program code, stored on the computer-readable medium, the computer-readable program code having instructions, which when executed cause a controller operably coupled to a medication infusion pump to administer the interferon-α to a patient infected with the hepatitis C virus according to a patient-specific therapeutic regimen made by:
administering interferon-α to the patient following a first therapeutic regimen;
obtaining pharmacokinetic or pharmacodynamic parameters from the patient so as to observe a patient-specific response to the first therapeutic regimen wherein the pharmacokinetic or pharmacodynamic parameters comprise at least one of:
a concentration of interferon-α in the blood of the patient that results from the first therapeutic regimen; or
a concentration of hepatitis C virus present in the patient;
using the pharmacokinetic or pharmacodynamic parameters observed in the patient in response to the first therapeutic regimen to obtain a patient-specific regimen responsiveness profile; and
using the patient-specific regimen responsiveness profile to make the patient-specific therapeutic regimen.
